(19) United States
(12) Patent Application Publication
(10) Pub. No.: US 2026/0183160 A1
(43) Pub. Date: Jul. 2, 2026

(54) REFASTENABLE ABSORBENT ARTICLES WITH IMPROVED FASTENING

(71) Applicant: The Procter & Gamble Company, Cincinnati, OH (US)

(72) Inventors: Koichi MORIMOTO, Cincinnati, OH (US); Bin WANG, Beijing (CN); Ray Dennis DRIA, Mason, OH (US); Qin LI, Mason, OH (US)

(21) Appl. No.: 19/551,840

(22) Filed: Feb. 27, 2026

Related U.S. Application Data
(63) Continuation of application No. PCT/CN2023/123218, filed on Oct. 7, 2023.
(60) Provisional application No. 63/540,448, filed on Sep. 26, 2023.

(51) Int. Cl. A61F 13/56; A61F 13/15; A61F 13/49; A61F 13/496; A61F 13/58

(52) U.S. Cl. CPC A61F 13/5655; A61F 13/49011; A61F 13/496; A61F 2013/15284; A61F 2013/49025; A61F 2013/5677; A61F 2013/588

(57) ABSTRACT

Absorbent articles having a chassis and a first waist region and a second waist region. The first waist region joined to a first chassis end region and the second waist region joined to a second chassis end region. The first waist region having a first surface and an opposing second surface. The first and second end regions of the first waist region each include a fastener component disposed on the first surface of the first waist region. The fastener components each comprise a plurality of hooks. The first waist region comprises a total fastener caliper measured from the second surface of the first waist region to a distal end of the plurality of hooks of from 0.3 mm to 1.2 mm. The fastener components exhibit a Peak Peel Strength of 2.0 N/inch or more.

### CROSS REFERENCE TO RELATED APPLICATION

This application is a continuation of Patent Application No. PCT/CN2023/123218, filed on Oct. 7, 2023, which claims the benefit of U.S. Provisional Application No. 63/540,448, filed on Sep. 26, 2023, which are incorporated herein by reference.

### FIELD

The present disclosure relates to absorbent articles, and more particularly, to refastenable absorbent articles with improved fastening.

### BACKGROUND

Infants and other incontinent individuals wear absorbent articles to receive and contain urine and other body fluids such as feces. Some caregivers may prefer refastenable disposable absorbent articles with an underwear-like look and feel (e.g., soft, cloth-like, etc.) to aid in toilet training. The more of an underwear-like look and feel that an absorbent article possesses, the more likely that a child will be willing to accept utilizing the product during the toilet training process.

Disposable absorbent articles are often manufactured with fastener components, such as hooks, positioned on laterally opposing ends of the front or back waist region. The opposing waist region may have a cooperating area of material (often called a “landing zone”) with which the hooks are designed to engage to effect attachment therebetween and enable fastening of the article about the wearer's waist. In order to provide secure attachment between the hooks and the landing zone, it may be desirable that the hooks be relatively thick and stiff. However, such hooks can cause red markings or irritation of the wearer's skin and can disrupt the desired soft, cloth-like feel of the article. On the other hand, thinner, more flexible hooks may fail to provide a sufficient peel force for secure engagement between the hooks and landing zone during wear. This can compromise fastening performance, resulting in improper fit of the article about the wearer and/or an increase in leaks.

There is a need for disposable refastenable absorbent articles that provide secure attachment of the front and back waist regions yet still deliver an underwear-like look and feel.

### SUMMARY

The present disclosure solves the problem of the current stiff and uncomfortable absorbent article hooks by providing an improved fastener component that securely fastens the absorbent article around the wearer while delivering a more underwear-like look and feel.

Described herein is an absorbent article comprising a chassis comprising a topsheet, a backsheet, and an absorbent core positioned between the topsheet and the backsheet. The chassis further comprises a first chassis end region, a second chassis end region longitudinally separated from the first chassis end region by a crotch region, a first chassis side region, and a laterally opposing second chassis side region. The absorbent article further comprises a first waist region joined to the first chassis end region, and a second waist region joined to the second chassis end region. The first waist region comprises a first end region and a second end region laterally separated from the first end region by a central region. The first waist region further comprises a first surface and a second surface opposite the first surface. The second waist region comprises a first end region and a laterally opposed second end region. The first end region of the first waist region comprises a first fastener component disposed on the first surface, and the second end region of the first waist region comprises a second fastener component disposed on the first surface. The first fastener component is refastenably connected with the first end region of the second waist region and the second fastener component is refastenably connected with the second end region of the second waist region. The first and second fastener components each comprise a plurality of hooks. The first waist region comprises a total fastener caliper measured from the second surface of the first waist region to a distal end of the plurality of hooks of from 0.3 mm to 1.2 mm. The fastener components exhibit a Peak Peel Strength of 2.0 N/inch or more.

### BRIEF DESCRIPTION OF THE DRAWINGS

FIG. 1 shows a perspective view of a refastenable diaper pant in a pre-fastened configuration.

FIG. 2A shows a top view of the diaper pant of FIG. 1.

FIG. 2B shows a top view of the diaper pant of FIG. 2A in a compressed state.

FIG. 2C1 is a cross-sectional view of the diaper pant of FIG. 2B taken along line 2C-2C showing details of a flange connection with a first configuration of belt laminate structures.

FIG. 2C2 is a cross-sectional view of the diaper pant of FIG. 2B taken along line 2C-2C showing details of a flange connection with a second configuration of belt laminate structures.

FIG. 2C3 is a cross-sectional view of the diaper pant of FIG. 2B taken along line 2C-2C showing details of a flange connection with a third configuration of belt laminate structures.

FIG. 2C4 is a cross-sectional view of the diaper pant of FIG. 2B taken along line 2C-2C showing details of a flange connection with a fourth configuration of belt laminate structures with the second belt provided with a stiffening patch.

FIG. 2C5 is a cross-sectional view of the diaper pant of FIG. 2B taken along line 2C-2C showing details of a flange connection with a fifth configuration of belt laminate structures with the second belt provided with a stiffening patch.

FIG. 2C6 is a cross-sectional view of the diaper pant of FIG. 2B taken along line 2C-2C showing details of a flange connection with a sixth configuration of belt laminate structures with the second belt provided with a stiffening patch.

FIG. 2D is a detailed sectional view of a bond structure between substrates of the flange and first belt comprising substantially tackifier free adhesive.

FIG. 2E is a detailed view of the bond structure of FIG. 2D applied between two nonwoven substrates.

FIG. 3A shows a plan view of a diaper pant with the portion of the diaper that faces away from a wearer oriented toward the viewer.

FIG. 3B shows a plan view of a diaper pant with the portion of the diaper that faces toward a wearer oriented toward the viewer.

FIG. 3C shows a plan view of a diaper pant with the portion of the diaper that faces away from a wearer oriented toward the viewer, illustrating example elastic material arrangements in the first and second belts.

FIG. 3D shows a plan view of a diaper pant with the portion of the diaper that faces away from a wearer oriented toward the viewer, illustrating first and second belt size and shape features.

FIG. 3E shows a plan view of a diaper pant with the portion of the diaper that faces away from a wearer oriented toward the viewer, illustrating first and second belt size and shape features.

FIG. 3F shows a plan view of a diaper pant with the portion of the diaper that faces away from a wearer oriented toward the viewer, illustrating first and second belt size and shape features.

FIG. 4 is a cross-sectional view of the diaper pant of FIG. 3A taken along line 4-4 showing first and second elastic belts provided with panel layers.

FIG. 4A is a cross-sectional detailed view of a first belt provided with panel layers wherein one panel layer is folded over another panel layer.

FIG. 4A1 is a cross-sectional detailed view of another example configuration wherein the first belt is provided with panel layers wherein one panel layer is folded over another panel layer.

FIG. 4A2 is a cross-sectional detailed view of another example configuration wherein the first belt is provided with panel layers wherein one panel layer is folded over another panel layer.

FIG. 4B is a cross-sectional detailed view of a second belt provided with panel layers wherein one panel layer is folded over another panel layer.

FIG. 5A shows a perspective view of a diaper pant with a continuous outer cover in a pre-fastened configuration.

FIG. 5B shows a plan view of a diaper pant with a continuous outer cover with the portion of the diaper that faces away from a wearer oriented toward the viewer.

FIG. 5C is a cross-sectional view of the diaper pant of FIG. 5B taken along line 5C-5C showing first and second elastic belts provided with panel layers and a continuous outer cover.

FIG. 5D is a cross-sectional view of a diaper pant of showing first and second elastic belts provided with panel layers formed with a continuous inner layer and a continuous outer cover.

FIG. 6A is a perspective view of the diaper pant of FIG. 1 showing a first end region of the second belt partially disconnected from a first flange.

FIG. 6B is a perspective view of the diaper pant of FIG. 6A showing a second end region of the second belt partially disconnected from a second flange.

FIG. 7A is a detailed view of a diaper pant showing a flange bonded with a first belt and refastenably connected with a second belt.

FIG. 7B is a detailed view of the diaper pant of FIG. 7A showing the second belt partially disconnected from the flange.

FIG. 7C is a detailed view of the diaper pant of FIG. 7B showing the second belt completely disconnected from the flange.

FIG. 8 is a planar view of a fastener component on a flange.

FIG. 9A is a cross-sectional view of an example configuration of the flange and fastener component of FIG. 8 taken along line 9-9 showing a base of the fastener component bonded with the flange.

FIG. 9B is a cross-sectional view of an example configuration of the flange and fastener component of FIG. 8 taken along line 9-9 showing a base of the fastener component extrusion bonded with a backing layer that is bonded with the flange.

FIG. 9C is a cross-sectional view of an example configuration of the flange and fastener component of FIG. 8 taken along line 9-9 showing a base of the fastener component extrusion bonded with the flange.

FIG. 9D is a cross-sectional view of an example configuration of the flange and fastener component of FIG. 8 taken along line 9-9 showing a base of the fastener component extrusion bonded with the flange.

FIG. 9E is a cross-sectional view of an example configuration of the flange and fastener component of FIG. 8 taken along line 9-9 showing a fastener component comprising hooks formed directly from material of the flange.

FIG. 10A is a detailed view of a diaper pant showing a belt directly refastenably connected with a fastener component on a flange.

FIG. 10B is a detailed view of a diaper pant showing a fastener component on a belt directly refastenably connected with a flange.

FIG. 10C is a detailed view of a diaper pant showing a fastener component comprising loops on a belt refastenably connected with a fastener component comprising hooks on a flange.

FIG. 10D is a detailed view of a diaper pant showing a fastener component comprising hooks on a belt refastenably connected with a fastener component comprising loops on a flange.

FIG. 11A is a cross-sectional view of a first configuration of a fastener component on a first waist region showing a base of the fastener component bonded with the first waist region.

FIG. 11B is a cross-sectional view of a second configuration of a fastener component on a first waist region showing a fastener component comprising hooks formed directly from material of the first waist region.

FIG. 12 is a side perspective view of a refastenable diaper pant comprising side panels where the fastener component is shown engaged on one side of the diaper pant and disengaged on the other side of the diaper pant.

FIGS. 13A-C are a test method arrangement for the Fastener Ultra Sensitive 3 Point Bending Method.

FIG. 14 is a schematic view of an example of a hanger-type sample holding fixture according to the “Whole Article Force Measurement”.

### DETAILED DESCRIPTION

### Definitions

The following term explanations may be useful in understanding the present disclosure:

“Absorbent article” refers to devices, which absorb and contain body exudates and, more specifically, refers to devices, which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, menstrual pads and the like.

“Body-facing” and “garment-facing” refer respectively to the relative location of an element or a surface of an element or group of elements. “Body-facing” (also referred to herein as wearer facing) implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

The terms “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force. Elastomeric materials may include elastomeric films, scrims, nonwovens, ribbons, strands and other sheet-like structures.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “distal” is used to describe a position situated away from a center of a body or from a point of attachment, and the term “proximal” is used to describe a position situated nearer to a center of a body or a point of attachment.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

“Pre-strain” refers to the strain imposed on an elastic or elastomeric material prior to combining it with another element of the elastomeric laminate or the absorbent article. Pre-strain is determined by the following equation Pre-strain=((extended length of the elastic-relaxed length of the elastic)/relaxed length of the elastic)*100.

“Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897; 5,360,420; 5,599,335; 5,643,588; 5,674,216; 5,702,551; 5,968,025; 6,107,537; 6,118,041; 6,153,209; 6,410,129; 6,426,444; 6,586,652; 6,627,787; 6,617,016; 6,825,393; and 6,861,571; and U.S. Patent Publication Nos. 2013/0072887 A1; 2013/0211356 A1; and 2013/0306226 A1, all of which are incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, back waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1; 2012/0061016 A1; 2012/0061015 A1; 2013/0255861 A1; 2013/0255862 A1; 2013/0255863 A1; 2013/0255864 A1; and 2013/0255865 A1, all of which are incorporated by reference herein.

“Closed-form” means opposing waist regions are joined, as packaged, either permanently or refastenably to form a continuous waist opening and leg openings.

“Open-form” means opposing waist regions are not initially joined to form a continuous waist opening and leg openings but comprise a closure means such as a fastening system to join the waist regions to form the waist and leg openings before or during application to a wearer of the article.

“Inboard,” with respect to a first feature of an article and its position relative a second feature or location on the article, means that the first feature lies closer to a respective axis of the article than the second feature or location, along a horizontal x-y plane approximately occupied by the article when laid out flat, extended to the full longitudinal and lateral dimensions of its component web materials against any contraction induced by any included pre-strained elastomeric material, on a horizontal surface. Laterally inboard means the first feature is closer to the longitudinal axis, and longitudinally inboard means the first feature is closer to the lateral axis. Conversely, “outboard,” with respect to a first feature of an article and its position relative a second feature or location on the article, means that the first feature lies farther from the respective axis of the article than the second feature or location.

The present disclosure relates to absorbent articles, and more particularly, to absorbent articles having refastenably connected first and second waist regions. In some configurations, an absorbent article may comprise: a chassis comprising a topsheet, a backsheet, and an absorbent core positioned between the topsheet and the backsheet. The chassis may further comprise a first chassis end region and a second chassis end region longitudinally separated from the first end region by a crotch region. The absorbent article may comprise a first waist region joined to the first chassis end region and a second waist region joined to the second chassis end region. The first waist region and the second waist region may each comprise a first end region and a second end region laterally separated from the first end region by a central region. The first waist region may further comprise a first surface and a second surface opposite the first surface. The first and second end regions of the first waist region may each comprise a fastener component disposed on the first surface. The fastener component may be configured to refastenably engage with a material of the opposing end region of the second belt. As discussed below, the fastener components may be configured to be thin and flexible yet still provide a sufficient peel force to help keep the article securely fastened about the wearer's waist during use.

In some configurations, the first waist region may comprise a first belt and the second waist region may comprise a second belt, each belt comprising a garment facing surface and an opposing wearer facing surface and each belt comprising a first end region and a second end region laterally separated from the first end region by a central region. The first chassis end region may be connected with the central region of the first belt, and the second chassis end region may be connected with the central region of the second belt. Each belt may further comprise an outer laterally extending edge and a longitudinally opposed inner laterally extending edge. The absorbent article may also comprise a first flange and a second flange, each flange comprising a first surface and a second surface opposite the first surface, and each flange comprising a first end region and a second end region. The first surface of the first end region of the first flange may be bonded with the wearer facing surface of the first end region of the first belt, and the second surface of the second end region of the first flange may be in a facing relationship with the wearer facing surface of the second belt. The second end region of the first flange may be refastenably connected with the first end region of the second belt. In addition, the first surface of the first end region of the second flange may be bonded with the wearer facing surface of the second end region of the first belt, and the second surface of the second end region of the second flange may be in a facing relationship with the wearer facing surface of the second belt. The second end region of the second flange may be refastenably connected with the second end region of the second belt.

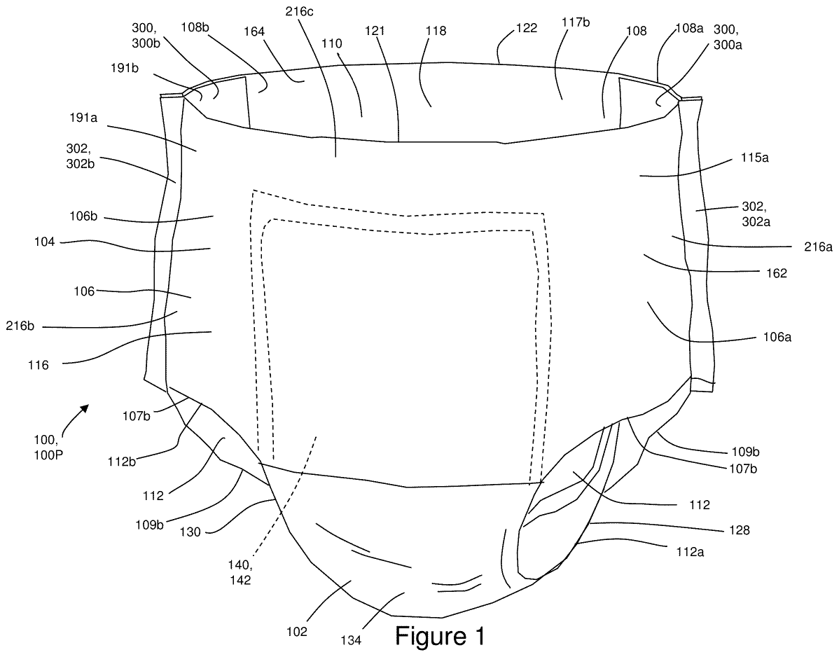

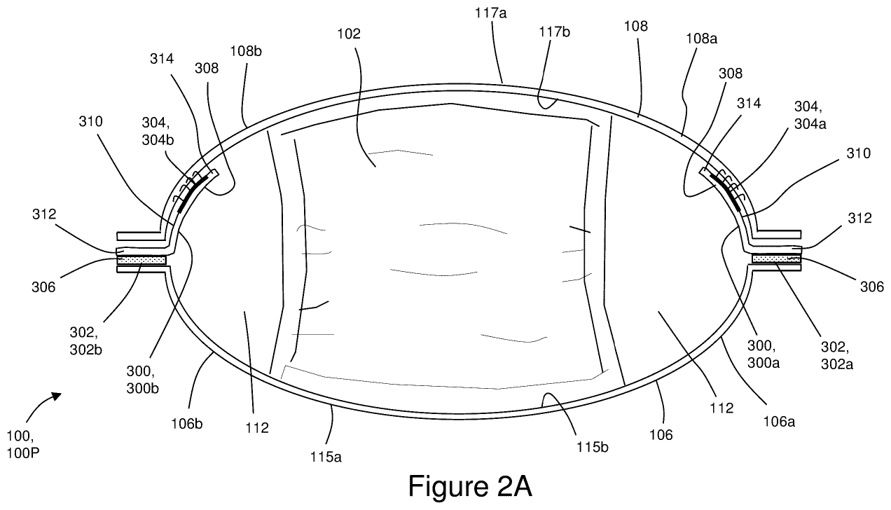

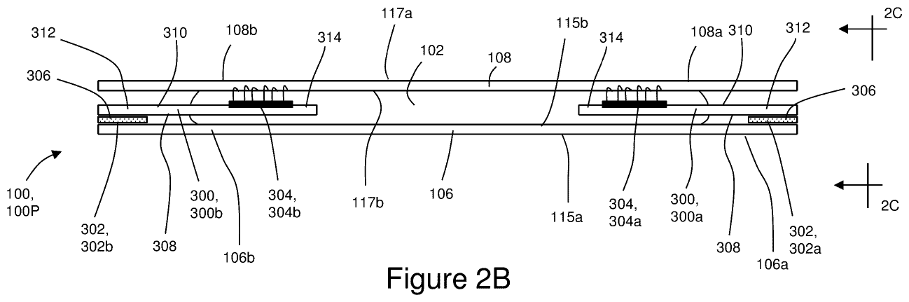

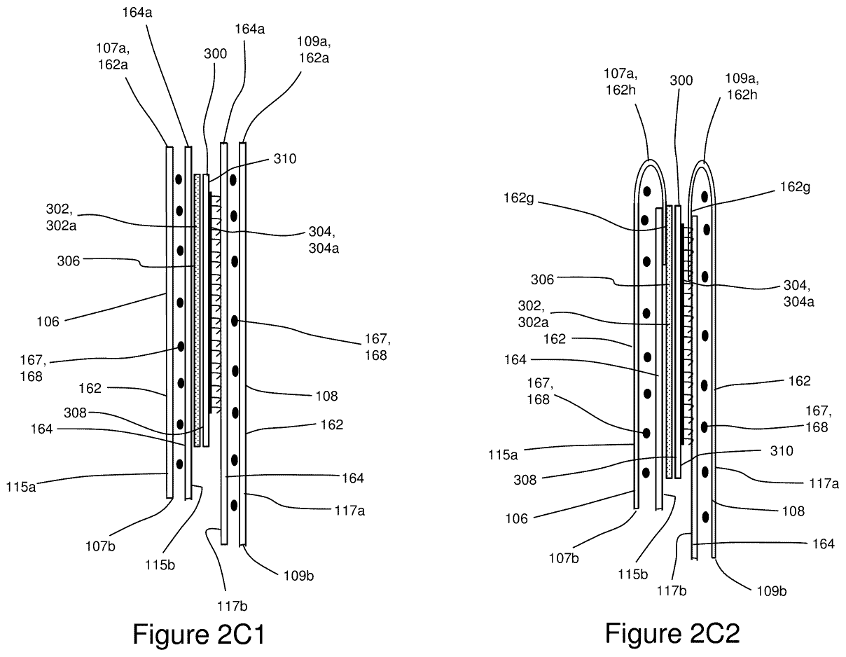

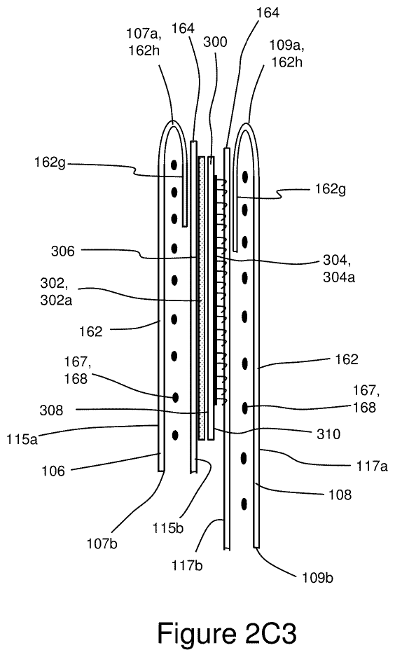

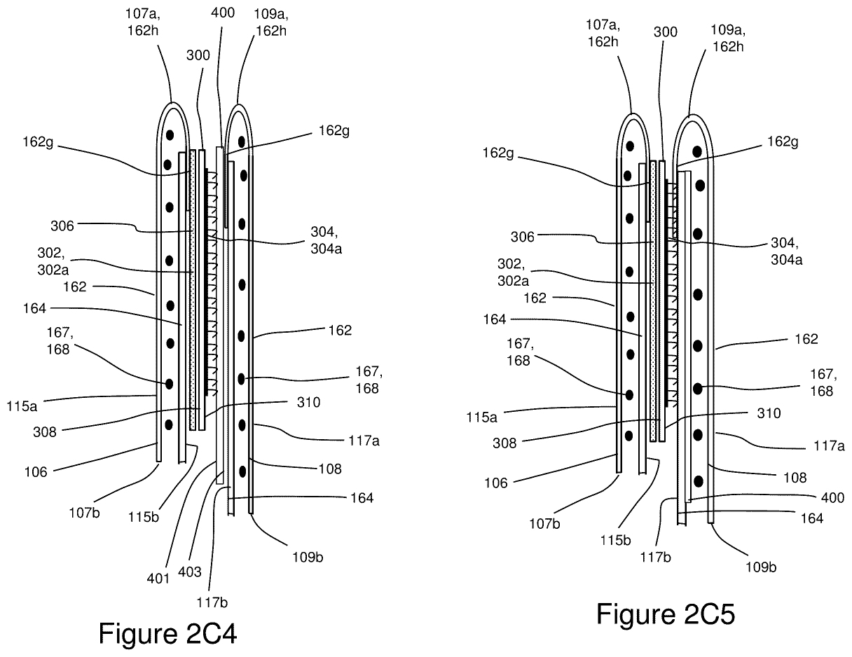

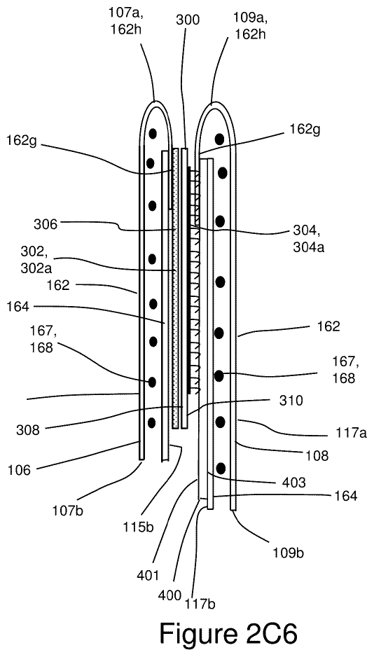

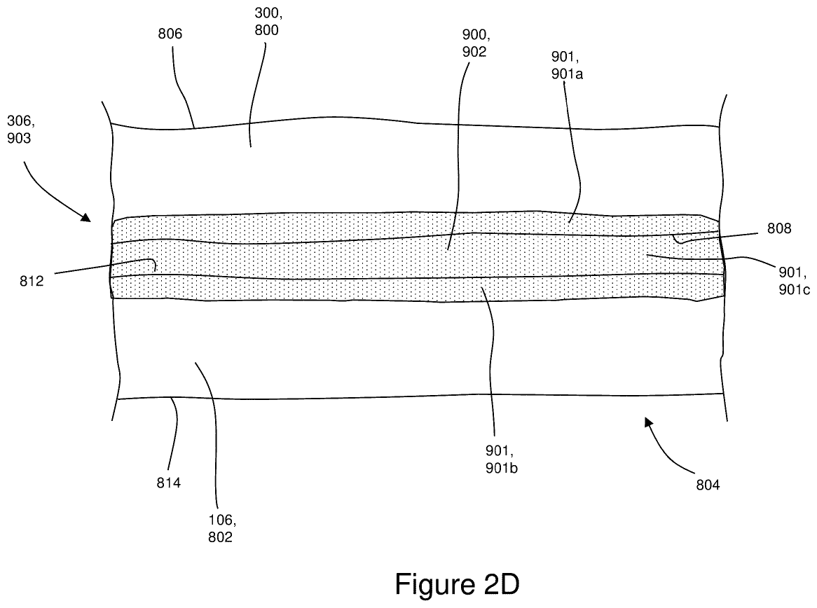

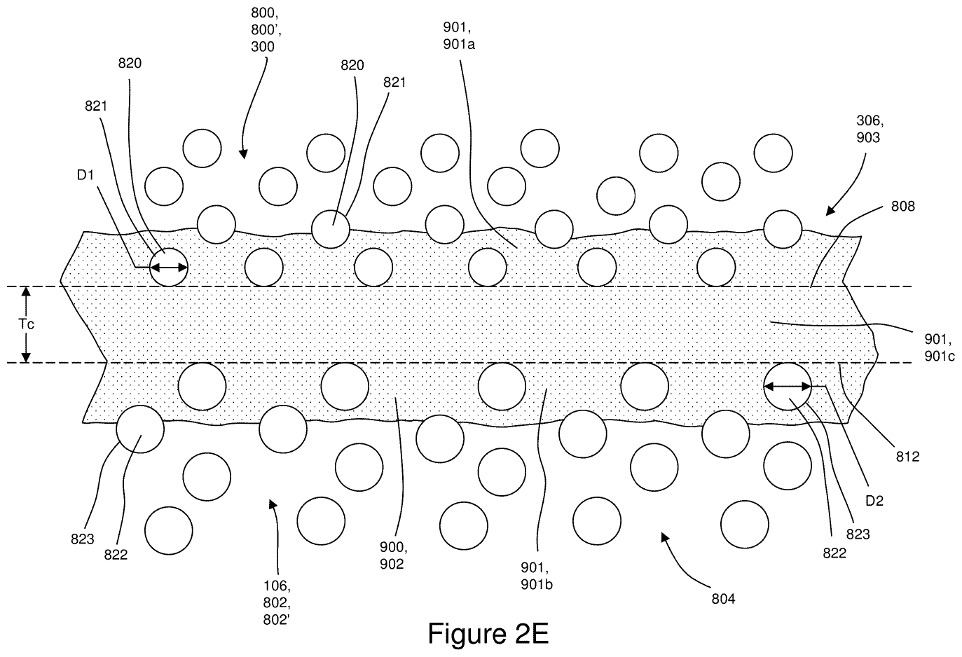

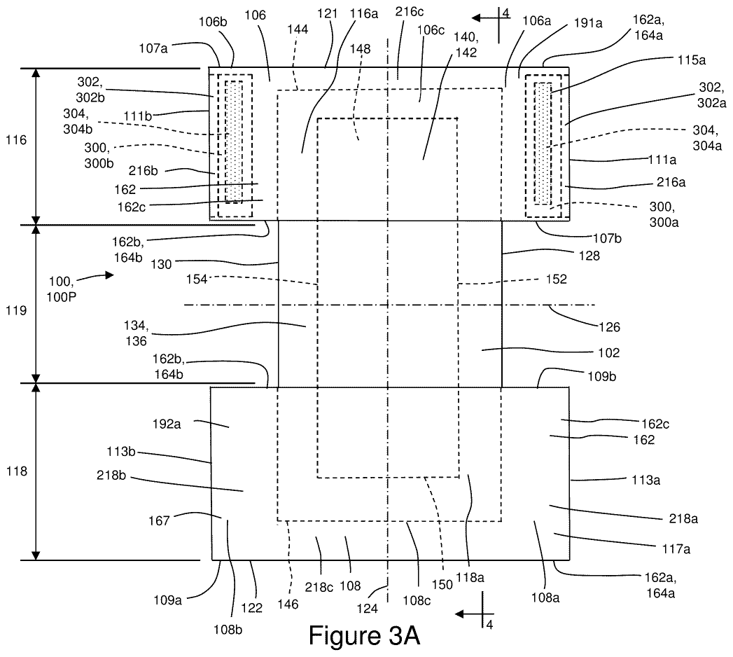

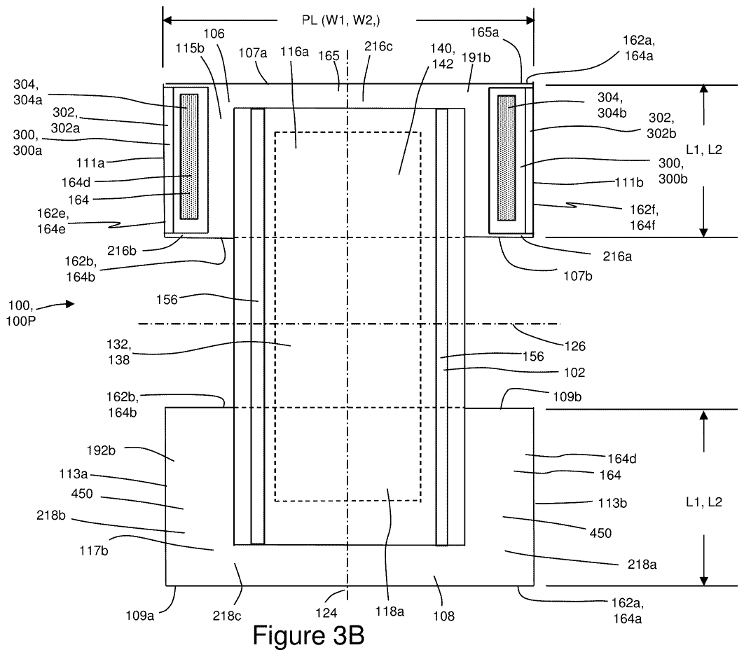

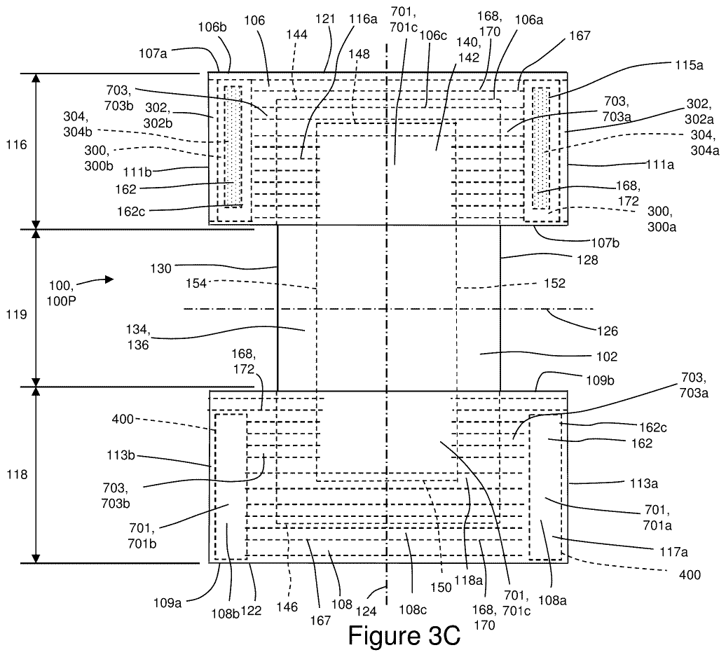

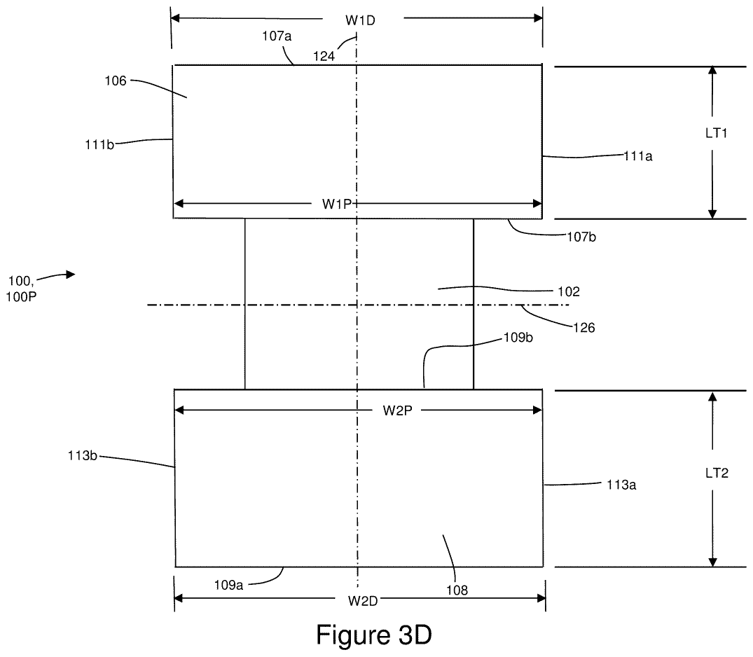

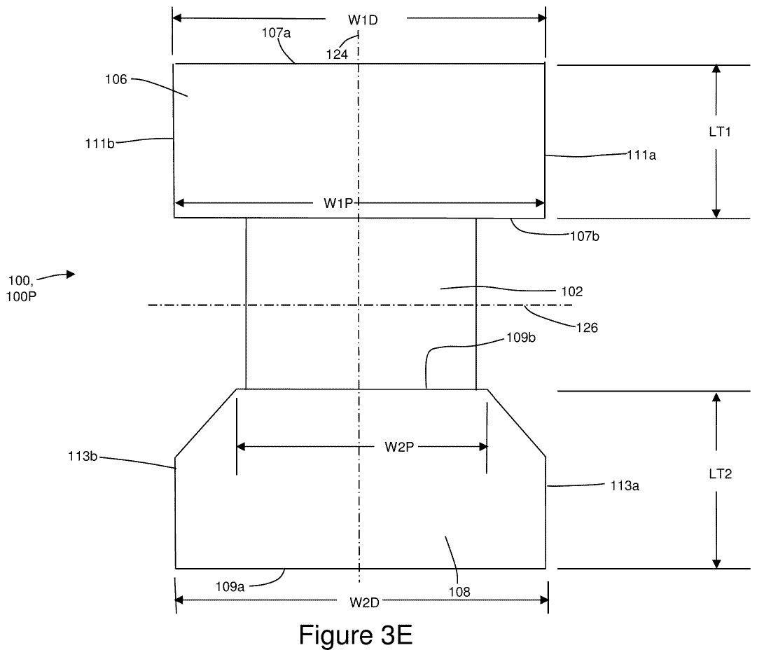

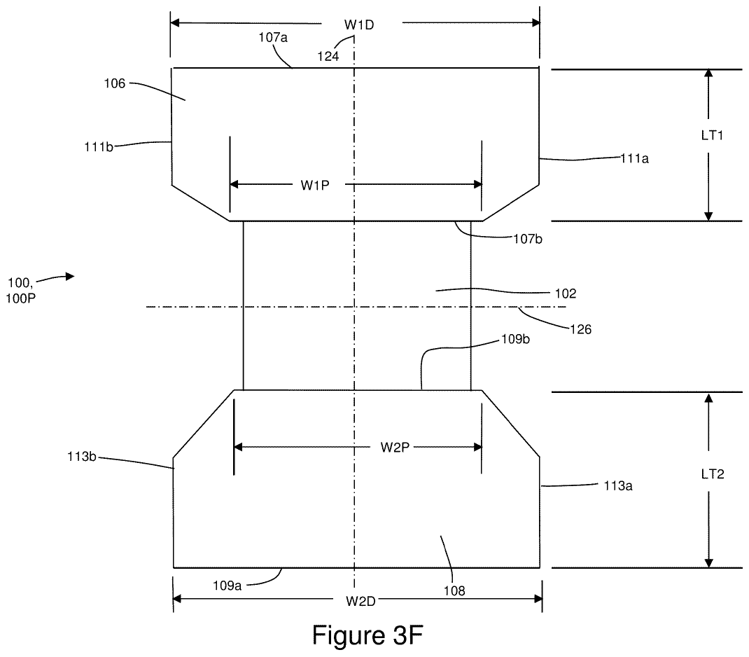

FIGS. 1-3B show an example of an absorbent article 100 in the form of a diaper pant 100P that may include components constructed in accordance with the configurations disclosed herein. In particular, FIG. 1 shows a perspective views of a diaper pant 100P in a pre-fastened configuration. FIG. 2A shows a top view of the diaper pant of FIG. 1, and FIG. 2B shows a top view of the diaper pant of FIG. 2A in a compressed state. FIG. 3A shows a plan view of the diaper pant 100P with the portion of the diaper that faces away from a wearer oriented toward the viewer, and FIG. 3B shows a plan view of the diaper pant 100P with the portion of the diaper that faces toward a wearer oriented toward the viewer. The diaper pant 100P includes a chassis 102 and a ring-like elastic belt 104. As discussed below in more detail, a first elastic belt 106 (also referred to herein as first belt) and a second elastic belt 108 (also referred to herein as second belt) are refastenably connected together to form the ring-like elastic belt 104.

With continued reference to FIGS. 1-3B, the diaper pant 100P and the chassis 102 each include a first waist region 116, a second waist region 118, and a crotch region 119 disposed intermediate the first and second waist regions. It may also be described that the chassis 102 includes a first chassis end region 116a, a second chassis end region 118a, and a crotch region 119 disposed intermediate the first and second chassis end regions 116a, 118a. The first waist region 116 may be configured as a front waist region, and the second waist region 118 may be configured as back waist region.

The first waist region 116 may comprise a first end region 216a and a second end region 216b laterally separated from the first end region 216a by a central region 216c. The second waist region 118 may comprise a first end region 218a and a second end region 218b laterally separated from the first end region 218a by a central region 218c. The first waist region 116 may be joined to the first chassis end region 116a and the second waist region may be joined to the second chassis end region 118a. The first and second waist regions may have a basis weight of from about 25 gsm to about 90 gsm, or from about 30 gsm to about 75 gsm, or from about 35 gsm to about 65 gsm. The first waist region 116 may further comprise a first surface 191a and a second surface 191b opposite the first surface 191a. The second waist region 118 may also comprise a first surface 192a and a second surface 192b opposite the first surface 192a.

In some configurations, the first and second end regions 216a, 216b of the first waist region 116 may each comprise a fastener component 304 disposed on the first surface 191a. The fastener components 304 may be configured to refastenably connect with a landing zone 450 on the second surface 192b of the first and second end regions 218a, 218b of the second waist region 118. In some configurations, the landing zone 450 may be or may comprise one or more discrete nonwoven materials that are attached to a portion of the second waist region 118. The landing zone 450 may comprise a material, such as an adhered patch of loop material or an adhered patch of nonwoven material that is adapted for use in providing refastenable engagement and suitable attachment strength when engaged with hooks. Examples of such nonwoven materials are described in, for example, U.S. Pat. No. 7,789,870. In some configurations, nonwoven materials used as components of the second waist region, such as a substrate of an elastic belt, may be deemed suitably lofty and to have fibers that are dense, strong enough, and sufficiently bonded within the material, to sufficiently engage with suitably designed hooks, and provide sufficient attachment strength, and thus no discrete patch of landing zone material may be needed.

The diaper 100P may also include a laterally extending front waist edge 121 (also referred to herein as a first waist edge) in the first waist region 116 and a longitudinally opposing and laterally extending back waist edge 122 (also referred to herein as a second waist edge) in the back waist region 118 (also referred to as a second waist region). To provide a frame of reference for the present discussion, the diaper 100P and chassis 102 of FIGS. 3A and 3B are shown with a longitudinal axis 124 and a lateral axis 126. In some configurations, the longitudinal axis 124 may extend through the front waist edge 121 and through the back waist edge 122. And the lateral axis 126 may extend through a first longitudinal or right side edge 128 and through a second longitudinal or left side edge 130 of the chassis 102. As previously mentioned, the longitudinal axis 124 extends perpendicularly through the front waist edge 121 and the back waist edge 122, and the lateral axis 126 extends perpendicularly to the longitudinal axis 124. When the diaper pant 100P is worn, the longitudinal direction may extend from the wearer's front waist, through the crotch, to the wearer's back waist.

Referring to FIGS. 1-3B, the diaper pant 100P may comprise an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100P may also include other features, such as leg elastics and/or leg cuffs to enhance the fit around the legs of the wearer.

As shown in FIG. 3A, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 3A, the laterally extending end edges 144 and 146 may be located longitudinally inward from the laterally extending front waist edge 121 in the first waist region 116 and the laterally extending back waist edge 122 in the second waist region 118. In some configurations, the laterally extending end edges 144 and 146 may be coterminous with or located longitudinally outward from the laterally extending front waist edge 121 in the first waist region 116 and the laterally extending back waist edge 122 in the second waist region 118. When the diaper pant 100P is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the first waist region 116 through the crotch region 119 to the second waist region 118.

As previously mentioned, the diaper pant 100P may include a backsheet 136. The backsheet 136 may also define the outer, garment facing surface 134 of the chassis 102. The backsheet 136 may also comprise a woven or nonwoven material, polymeric films such as thermoplastic films of polyethylene or polypropylene, and/or a multi-layer or composite materials comprising a film and a nonwoven material. The backsheet may also comprise an elastomeric film. An example backsheet 136 may be a polyethylene film having a thickness of from about 0.012 mm (0.5 mils) to about 0.051 mm (2.0 mils). Further, the backsheet 136 may permit vapors to escape from the absorbent core (i.e., the backsheet is breathable) while still preventing exudates from passing through the backsheet 136.

Also described above, the diaper pant 100P may include a topsheet 138. The topsheet 138 may also define all or part of the inner, wearer facing surface 132 of the chassis 102. The topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art. Topsheets 138 may be selected from high loft nonwoven topsheets, apertured film topsheets and apertured nonwoven topsheets. Exemplary apertured films may include those described in U.S. Pat. Nos. 5,628,097; 5,916,661; 6,545,197; and 6,107,539, all of which are incorporated by reference herein.

As mentioned above, the diaper pant 100P may also include an absorbent assembly 140 that is joined to the chassis 102. As shown in FIG. 3A, the absorbent assembly 140 may have a laterally extending front edge 148 in the first waist region 116 and may have a longitudinally opposing and laterally extending back edge 150 in the second waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735, all of which are incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein.

As previously mentioned, the diaper 100P may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1, all of which are incorporated by reference herein.

In some configurations, the diaper pants may be manufactured with a ring-like elastic belt 104 and provided to consumers in a configuration wherein the first waist region 116 and the second waist region 118 are connected to each other as packaged, prior to being applied to the wearer. As such, diaper pants may have a continuous perimeter waist opening 110 and continuous perimeter leg openings 112 such as shown in FIG. 1. The ring-like elastic belt may be formed by joining a first elastic belt to a second elastic belt with an openable and reclosable fastening system disposed at or adjacent the laterally opposing sides of the belts.

The ring-like elastic belt 104 may be defined by a first elastic belt 106 (also referred to herein as a first belt) connected with a second elastic belt 108 (also referred to herein as a second belt). In some configurations, a portion of the first waist region 116 may comprise the first elastic belt 106. In some configurations, the second waist region 118 may comprise the second elastic belt 108. The first waist region 116 may extend laterally across the entire chassis 102, wherein the first chassis end region 116a may be connected with the central region 216c of the first waist region 116, and the second chassis end region 118a may be joined to the central region 218c of the second waist region 118.

As shown in FIGS. 3A and 3B, the first elastic belt 106 extends between a first longitudinal side edge 111a and a second longitudinal side edge 111b and defines first and second opposing end regions 106a, 106b and a central region 106c. And the second elastic 108 belt extends between a first longitudinal side edge 113a and a second longitudinal side edge 113b and defines first and second opposing end regions 108a, 108b and a central region 108c. As measured in an extended state, the distance between the first longitudinal side edge 111a and the second longitudinal side edge 111b defines the pitch length, PL, of the first elastic belt 106, and the distance between the first longitudinal side edge 113a and the second longitudinal side edge 113b defines the pitch length, PL, of the second elastic belt 108. The central region 106c of the first elastic belt is connected with the first chassis end region 116a of the chassis 102, and the central region 108c of the second elastic belt 108 is connected with the second chassis end region 118a of the chassis 102.

In some configurations, the first waist region 116 may comprise flanges 300. As shown in FIGS. 1-2B, flanges 300 may be bonded with opposing end regions of the first belt 106 and may be refastenably connected with opposing end regions of the second belt 108 to define the ring-like elastic belt 104 as well as the waist opening 110 and leg openings 112. For example, a first flange 300a may be bonded with the first end region 106a of the first belt 106 at a first flange seam 302a, and a second flange 300b may be bonded with the second end region 106b of the first belt 106 at a second flange seam 302b. In turn, the first flange 300a may be refastenably connected with a landing zone 450 of the first end region 108a of the second belt 108, and the second flange 300b may be refastenably connected with a landing zone 450 the second end region 108b of the second belt 108. For example, as shown in FIGS. 1-2B, a first fastener component 304a on the first flange 300a may refastenably connect the first flange 300a with the second belt 108, and a second fastener component 304b on the second flange 300b may refastenably connect the second flange 300b with the second belt 108. In the configurations shown in FIGS. 2A and 2B, the fastener components 304 may be adapted to refastenably connect directly with the second belt 108. It is to be appreciated that various configurations of fastener components 304 may be located on the flanges 300, the first belt 106, and/or the second belt 108, as discussed in more detail below. It is also to be appreciated that in some configurations, flanges 300 may be bonded with the opposing end regions of the second belt 108 and may be adapted to refastenably connect with opposing end regions of the first belt 106. It is further to be appreciated that the first belt may be positioned in a front waist region or a back waist region, and the second belt may be positioned in a front waist region or a back waist region.

It is also to be appreciated that the fastener components 304 may be configured in various ways, such as hooks, loops, and/or adhesive. For example, the fastener components 304 may comprise hook elements or adhesive adapted to refastenably connect with another surface of the diaper pant 100P. In some configurations, the fastener component 304 may comprise loop elements adapted to refastenably connect with a hook surface on the diaper pant 100P. The fastener component 304 may be a separate element connected with the first or second waist regions in various ways, such as mechanical bonding, adhesive bonding, or both, or may be integrally formed from materials of the first or second waist regions. In some configurations, the fastener component 304 may be a separate element connected with the first belt 106, the second belt 108, and/or the flange 300. In some configurations, the fastener component 304 may be integrally formed from materials of the first belt 106, the second belt 108, and/or the flange 300. In some configurations, the flange 300 and/or fastener component 304 may be printed and/or comprise materials of various different colors to help enhance visibility from outside the diaper pant 100P.

It is to be appreciated that the flanges 300 may be constructed from various types of materials, such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some configurations, the flanges 300 may comprise various types of nonwovens, such as spunbond, carded, wet-laid, meltblown, hydroentangled. The flanges 300 may be configured to be stretchable or non-stretchable and/or hydrophilic or hydrophobic. In some configurations, the flanges 300 may be configured as a single layer of material or a laminate comprising two or more layers of material. In some configurations, the flanges 300 may have a basis weight of from about 25 gsm to about 90 gsm, or from about 30 gsm to about 75 gsm, or from about 35 gsm to about 50 gsm.

It is also to be appreciated that various types of bonds 306 such as illustrated in FIGS. 2A and 2B may be used to bond the flanges 300 with the first belt 106 at the flange seams 302. For example, the bonds 306 at the flange seams 302 may comprise mechanical, thermal, pressure, and/or adhesive bonds.

In some configurations, the bonds 306 may comprise a bond structure 403 that comprises substantially tackifier free adhesives or tackifier free adhesives, such as disclosed in U.S. Patent Publication No. US20200047420A1, which is incorporated by reference herein. The term “tackifier free adhesive” is used herein to refer to an adhesive composition comprising a polymer and/or a copolymer, wherein the adhesive composition is free of or devoid of tackifiers. Examples of such tackifier free adhesives are disclosed in U.S. Patent Publication Nos. US20190322900A1; US20190322901A1; US20190322909A1; US20190321241A1; US20190321242A1; and US20200108167A1, all of which are incorporated by reference herein. “Devoid of,” “free of,” and the like, as those terms are used herein, means that the adhesive composition does not have more than trace amounts of background levels of a given material, ingredient, or characteristic following these qualifiers; the amount of the material or ingredient does not cause harm or irritation that consumers typically associate with the material or ingredient; or the material or ingredient was not added to the adhesive composition intentionally. In some applications, “devoid of” and “free of” can mean there is no measurable amount of the material or ingredient. For example, the adhesive composition in some forms can contain no measurable amount of a tackifier. “Substantially tackifier free adhesive” is used herein to refer to an adhesive composition comprising a polymer and/or a copolymer, wherein the adhesive composition comprises less than 10% tackifiers by weight. As such, a “tackifier free adhesive” is also a “substantially tackifier free adhesive.” The term “tackifier” means those conventional tackifier resins commonly available in the adhesive art and industry that are used in typical hot melt adhesives. Examples of conventional tackifier resins include aliphatic hydrocarbon resins, aromatic modified aliphatic hydrocarbon resins, hydrogenated poly-cyclopentadiene resins, poly-cyclopentadiene resins, gum rosins, gum rosin esters, wood rosins, wood rosin esters, tall oil rosins, tall oil rosin esters, poly-terpene, aromatic modified poly-terpene, terpene-phenolic, aromatic modified hydrogenated poly-cyclopentadiene resins, hydrogenated aliphatic resins, hydrogenated aliphatic aromatic resins, hydrogenated terpene and modified terpene, and hydrogenated rosin esters.

When the flange 300 and the first belt 106 include nonwoven layers, penetration of the substantially tackifier free adhesive into the nonwovens may cause the substantially tackifier free adhesive to intermesh with and bond with fibers within the nonwovens to help strengthen bonds therebetween. FIG. 2D illustrates an example of a bond 306 comprising a bond structure 903 that comprises a substantially tackifier free adhesive 900 between a first substrate 800 of the flange 300 a second substrate 802 of the first belt 106. In particular, FIG. 2D shows an example of a detailed sectional view of a layer 901 of substantially tackifier free adhesive 900 after pressure has been exerted on the first and second substrates 800, 802 to form the bond structure 903 between the first and second substrates 800, 802. As shown in FIG. 2D, the bond 903 comprises a first portion 901a of the layer 901 of substantially tackifier free adhesive 900 that has penetrated into the first substrate 800, and a second portion 901b of the layer 901 of substantially tackifier free adhesive 900 that has penetrated into the second substrate 802. In particular, the first portion 901a of the layer 901 substantially tackifier free adhesive 900 has penetrated through a second surface 808 of first substrate 800 without reaching or exiting a first surface 806 of the first substrate 800. And the second portion 901b of the layer 901 of the substantially tackifier free adhesive 900 has penetrated through a first surface 812 of the second substrate 802 without reaching or exiting a second surface 814 of the second substrate 802. As such, the first portion 901a of the layer 901 of the substantially tackifier free adhesive 900 does not penetrate entirely through the thickness of the first substrate 800, and the second portion 901b of the layer 901 of the substantially tackifier free adhesive 900 does not penetrate entirely through the thickness of the second substrate 802. With continued reference to FIG. 2D, the bond 903 also comprises a central portion 901c of the layer 901, wherein the layer 901 of the substantially tackifier free adhesive 900 comprises a central portion 901c extends between the first portion 901a and the second portion 901b. As illustrated, the second surface 808 of the first substrate 800 and the first surface 812 of the second substrate 802 are separated from each other by the central portion 901c of the layer 901 of the substantially tackifier free adhesive 900.

As previously mentioned, the first substrate 800 may comprise a first nonwoven 800′ and/or the second substrate 802 may comprise a second nonwoven 802′ such as shown in FIG. 2E, wherein the portions of the bond 903 comprises the substantially tackifier free adhesive 900 that is intermeshed with fibers of the first nonwoven 800′ and the second nonwoven 802′. As shown in FIG. 2E, the first nonwoven 800′ may comprise first fibers 820, and the second nonwoven comprises second fibers 822. The first portion 901a of the layer 901 of substantially tackifier free adhesive 900 is intermeshed with the first fibers 820 at the second surface 808 of the first nonwoven 800′, and the second portion 901b of the layer 901 of the substantially tackifier free adhesive 900 is intermeshed with the second fibers 822 at first surface 812 of the second nonwoven 802′. As shown in FIG. 2E, one or more first fibers 820 at and/or adjacent the second surface 808 of the first nonwoven 800′ may comprise an outer perimeter 821 that is completely surrounded by the substantially tackifier free adhesive 900 of the first zone 901a of the layer 901. And one or more second fibers 822 at and/or adjacent the first surface 812 of the second nonwoven 802′ may comprise an outer perimeter 823 that is completely surrounded by the substantially tackifier free adhesive 900 of the second zone 901b of the layer 901. It is to be appreciated that the substantially tackifier free adhesive 900 may completely surround the outer perimeters 821 of additional first fibers 820 positioned away from the second surface 808 and further into the interior thickness of the first nonwoven 800′, and/or the substantially tackifier free adhesive 900 may completely surround the outer perimeters 823 of additional second fibers 822 positioned away from the first surface 812 and further into the interior thickness of the second nonwoven 802′. It is to be appreciated that the substantially tackifier free adhesive 900 may partially surround the outer perimeters 821 of additional first fibers 820 positioned away from the second surface 808 and further into the interior thickness of the first nonwoven 800′, and/or the substantially tackifier free adhesive 900 may partially surround outer perimeters 823 of additional second fibers 822 positioned away from the first surface 812 and further into the interior thickness of the second nonwoven 802′.

With continued reference to FIG. 2E, the first fibers 820 may comprise first diameters D1, and the second fibers 822 may comprise second diameters D2. The first diameters D1 may be the same or different than the second diameters D1. It is to be appreciated that the central portion 901c of the layer 901 of substantially tackifier free adhesive 900 may comprise a thickness Tc. In some configurations, the thickness Tc is greater than the first diameters D1 and/or the second diameters D2. In some configurations, the thickness Tc may be greater than 3 times the first diameters D1 and/or 3 times the second diameters D2.

Referring now to FIGS. 3A and 3B, the first elastic belt 106 also defines an outer laterally extending edge 107a and an inner laterally extending edge 107b, and the second elastic belt 108 defines an outer laterally extending edge 109a and an inner laterally extending edge 109b. The outer edge 107a of the first belt 106 is positioned longitudinally outward of the inner edge 107b, and the outer edge 109a of the second belt 108 is positioned longitudinally outward of the inner edge 109b. As such, as shown in FIG. 1, a perimeter edge 112a of one leg opening may be defined by portions of the inner laterally extending edge 107b of the first elastic belt 106, the inner laterally extending edge 109b of the second elastic belt 108, and the first longitudinal or right side edge 128 of the chassis 102. And a perimeter edge 112b of the other leg opening may be defined by portions of the inner laterally extending edge 107b, the inner laterally extending edge 109b, and the second longitudinal or left side edge 130 of the chassis 102. The outer laterally extending edges 107a, 109a may also define the front waist edge 121 and the laterally extending back waist edge 122 of the diaper pant 100P.

It is to be appreciated that the first elastic belt 106 and the second elastic belt 108 may define different sizes and shapes. In some configurations, the first elastic belt 106 and/or second elastic belt 108 may define curved contours. For example, the inner lateral edges 107b, 109b of the first and/or second elastic belts 106, 108 may include non-linear or curved portions in the first and second opposing end regions. Such curved contours may help define desired shapes to leg opening 112, such as for example, relatively rounded leg openings. In addition to having curved contours, the elastic belts 106, 108 may include elastic strands 168 that extend along non-linear or curved paths that may correspond with the curved contours of the inner lateral edges 1076, 109b.

FIG. 3D shows a configuration wherein the first elastic belt 106 and the second elastic belt 108 both define generally rectangular shapes. For example, as shown in FIG. 3D, the outer laterally extending edge 107a of the first elastic belt 106 may comprise a lateral width of WID and the inner laterally extending edge 107b may comprise a lateral width of WIP, wherein WID and WIP are equal or substantially equal. In addition, the outer laterally extending edge 109a of the second elastic belt 108 may comprise a lateral width of W2D and the inner laterally extending edge 109b may comprise a lateral width of W2P, wherein W2D and W2P are equal or substantially equal.

In some configurations, at least one of the first elastic belt 106 and the second elastic belt 108 may comprise lateral edges having different lengths. For example, FIG. 3E shows a configuration wherein the first elastic belt 106 defines a generally rectangular shape, such as described with reference to FIG. 3D, and wherein the outer laterally extending edge 109a of the second elastic belt 108 and the inner laterally extending edge 109b have different lengths. As shown in FIG. 3E, the outer laterally extending edge 109a of the second elastic belt 108 may comprise a lateral width of W2D and the inner laterally extending edge 109b may comprise a lateral width of W2P, wherein W2D is greater than W2P.

In some configurations, both the first elastic belt 106 and the second elastic belt 108 may comprise lateral edges having different lengths. For example, FIG. 3F shows a configuration wherein the outer laterally extending edge 107a of the first elastic belt 106 and the inner laterally extending edge 107b have different lengths, and wherein the outer laterally extending edge 109a of the second elastic belt 108 and the inner laterally extending edge 109b have different lengths. As shown in FIG. 3F, the outer laterally extending edge 107a of the first elastic belt 107 may comprise a lateral width of WID and the inner laterally extending edge 107b may comprise a lateral width of WIP, wherein WID is greater than WIP, and wherein the outer laterally extending edge 109a of the second elastic belt 108 may comprise a lateral width of W2D and the inner laterally extending edge 109b may comprise a lateral width of W2P, wherein W2D is greater than W2P.

With reference to FIGS. 3D-3F, the first elastic belt 106 may define a longitudinal length LT1 extending between outer laterally extending edge 107a and the inner laterally extending edge 107b, and the second elastic belt 108 may define a longitudinal length LT2 extending between outer laterally extending edge 109a and the inner laterally extending edge 109b. In some configurations, LT1 may be equal to LT2. In some configurations, LT1 may be less or greater than LT2. With continued reference to FIGS. 3D-3F, in some configurations, WID may be equal to WIP, or WID may be different than WIP. In some configurations, W2D may be equal to W2P, or W2D may be different than W2P. In some configurations, WID and/or WIP may be equal to or different W2D and/or W2P.

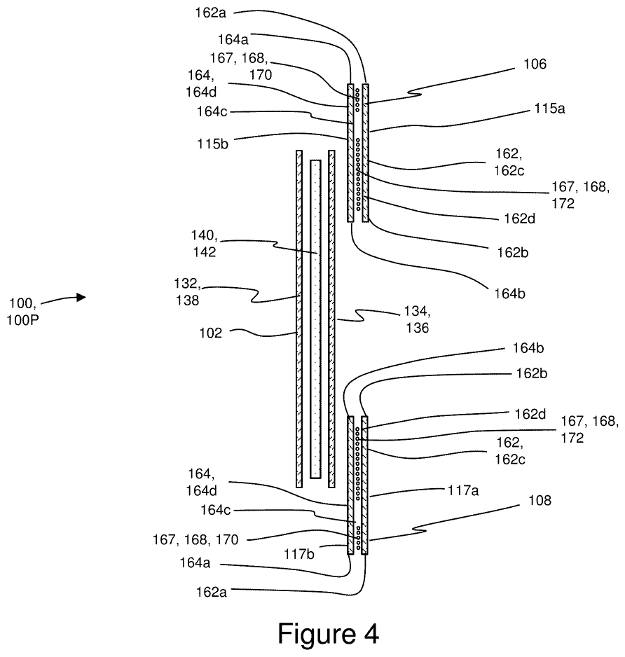

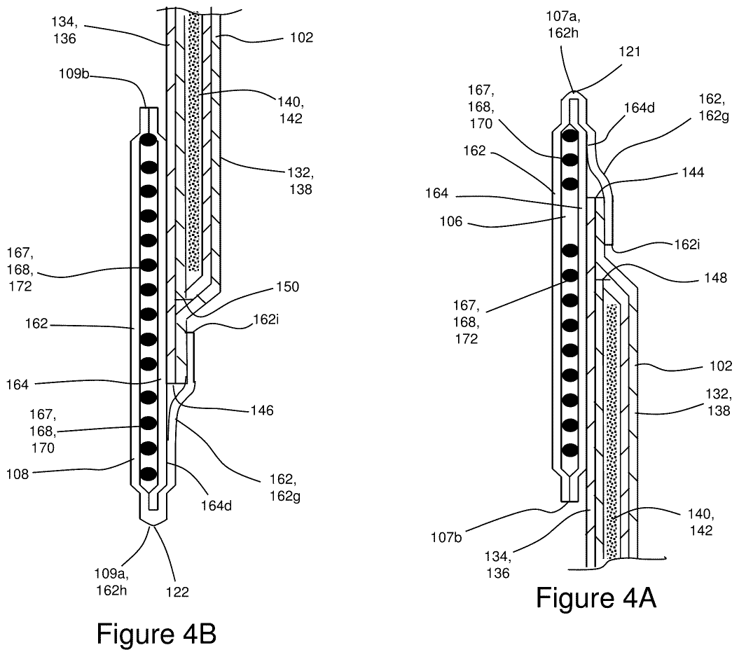

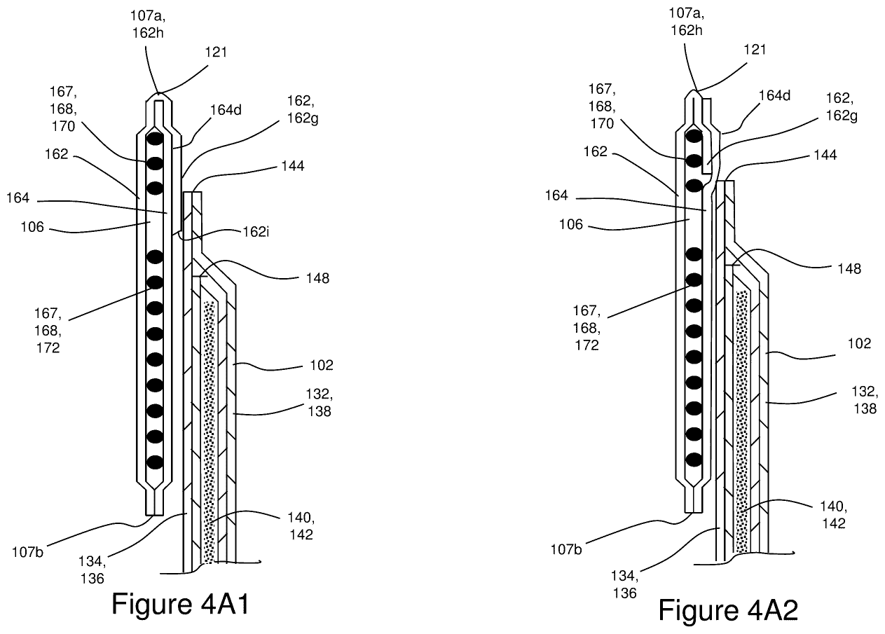

With reference to FIGS. 3A, 3B, and 4, the first elastic belt 106 and the second elastic belt 108 may also each include a first substrate 162 and a second substrate 164. The first substrates 162 may be oriented to define at least a portion of a garment facing surface 115a of the first elastic belt 106 and a garment facing surface 117a of the second elastic belt 108, and the second substrates 164 may be oriented to define at least a portion of a wearer facing surface 115b of the first elastic belt 106 and a wearer facing surface 117b of the second elastic belt 108. The first substrate 162 may extend from a proximal edge 162b to a distal edge 162a for a maximum length L1, and the second substrate 164 may extend from a proximal edge 164b to a distal edge 164a for a maximum length L2. It is to be appreciated that the distal edge 162a and/or the proximal edge 162b of the first substrate 162 may be straight and/or curved and/or may be parallel or unparallel to each other. It is also to be appreciated that the distal edge 164a and/or the proximal edge 164b of the second substrate 164 may be straight and/or curved and/or may be parallel or unparallel to each other. As such, the maximum length L1 refers to the longest distance extending longitudinally between the distal edge 162a and the proximal edge 162b of the first substrate 162, and the maximum length L2 refers to the longest distance extending longitudinally between the distal edge 164a and the proximal edge 164b of the second substrate 164. In some configurations, L1 may be equal to, less than, or greater than L2. In some configurations, L1 may be equal to or less than LT1, and L2 may be equal to or less than LT2. In some configurations, the distal edge 162a of the first substrate 162 may define at least a portion of the front waist edge 121 and/or at least a portion of back waist edge 122, and/or the distal edge 164a of the second substrate 164 may define at least a portion of the front waist edge 121 and/or at least a portion of back waist edge 122. As such, in some configurations, the distal edge 162a of the first substrate 162 and/or the distal edge 164a of the second substrate 164 may define at least a portion of the waist opening 110.

It is to be appreciated that the first substrate 162 and the second substrate 164 may define various lateral widths that may or may not be equal. For example, as shown in FIG. 3B, the first substrate 162 may extend laterally between a first longitudinal edge 162e and a second longitudinal edge 162f to define a first lateral width W1, and the second substrate 164 may extend laterally between a first longitudinal edge 164e and a second longitudinal edge 164f to define a second lateral width W2.

In some configurations, the proximal edge 162b of the first substrate 162 and/or the proximal edge 164b of the second substrate 164 may extend laterally across the backsheet 136. As shown in FIGS. 3A, 3B, and 4, the first substrate 162 includes a garment facing surface 162c and an opposing wearer facing surface 162d, and the second substrate 164 includes a garment facing surface 164c and an opposing wearer facing surface 164d.

In some configurations, the first elastic belt 106 and/or the second elastic belt 108 may include a folded portion of at least the first substrate 162 and/or the second substrate 164. For example, as shown in FIGS. 4A and 4B, the first elastic belt 106 and/or the second elastic belt 108 may include a folded portion 162g of the first substrate 162 extending longitudinally between a fold line 162h in the first substrate 162 and a lateral edge 162i. As such, the folded portion 162g of the first substrate 162 may be connected with the wearer facing surface 164d of the second substrate 164. In some configurations, the folded portion 162g of the first substrate 162 may also be connected with and/or overlap the chassis 102. In some configurations, the folded portion 162g of the first substrate 162 may also be connected with the wearer facing surface 162d of the first substrate 162. In some configurations, a portion of the folded portion 162g of the first substrate 162 may be left unbonded to the chassis 102 and/or the second substrate 164, forming a pocket having an opening oriented toward the lateral centerline 162c of the chassis 102. In another example, the first elastic belt 106 and/or the second elastic belt 108 may include a folded portion of the second substrate 164 extending longitudinally between a fold line in the second substrate 164 and a lateral edge. As such, the folded portion of the second substrate 164 may be connected with the garment facing surface 162c of the first substrate 162. As such, in some configurations, a fold line of the first substrate 162 and/or a fold line of the second substrate 164 may define at least a portion of the waist opening 110. It is to be appreciated that various waist configurations may be utilized. For example, as shown in FIG. 4A1, the folded portion 162g may be sandwiched between the second substrate 164 and the backsheet 136. In another example shown in FIG. 4A2, the second substrate 164 may be sandwiched between the folded portion 162g and the backsheet 136. Although FIGS. 4A1 and 4A2 show configurations of the first belt 106, it is to be appreciated that such configurations may be applied with the second belt 108.

It is to be appreciated that the first elastic belt 106 and the second elastic belt 108 may comprise the same materials and/or may have the same structure. In some embodiments, the first elastic belt 106 and the second elastic belt 108 may comprise different materials and/or may have different structures. It should also be appreciated that components of the first elastic belt 106 and the second elastic belt 108, such as the first substrate 162, and/or second substrate 164 may be constructed from various materials. For example, the first and/or second belts may include a first substrate 162, and/or second substrate 164 that may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some configurations, the first and/or second belts may include a first substrate 162, and/or second substrate 164 comprising a nonwoven web of synthetic fibers, and may include a stretchable nonwoven. In some configurations, the first and second elastic belts may include an inner hydrophobic, non-stretchable nonwoven material and an outer hydrophobic, non-stretchable nonwoven material. In some configurations, the first and/or second belts may include a first substrate 162 and/or second substrate 164 comprising a nonwoven material having a basis weight of from about 10 to about 35 gsm, or from about 15 to about 25 gsm. It is to be appreciated that the belts may configured in various ways, such as disclosed for example, in U.S. Patent Publication No. 2022/0142828A1, which is incorporated by reference.

Elastic material 167 may be positioned between the wearer facing surface 162d of the first substrate 162 and the garment facing surface 164c of the second substrate 164. It is to be appreciated that the elastic material 167 may include one or more elastic elements such as strands, ribbons, elastic films, or panels extending along the lengths of the elastic belts. As shown in FIGS. 3C and 4, the elastic material 167 may include a plurality of elastic strands 168. In some configurations, the elastic material 167 may be an elastic film used to form a zero-strain elastic laminate comprising an elastic film bonded to one or more nonwoven layers and subsequently subjected to mechanical deformation or activation sufficient to weaken the nonwoven layer(s) and enable the laminate to stretch and recover elastically.

It is also to be appreciated that the first substrate 162, second substrate 164, and/or elastic material 167 of the first elastic belt 106 and/or second elastic belt 108 may be bonded together and/or with other components, such as the chassis 102, with adhesive and/or mechanical bonds. It is to be appreciated that adhesive and mechanical bonding methods may be utilized alone or in combination with each other.

In some configurations, adhesive may be applied to at least one of the first substrate 162, second substrate 164, and/or elastic material 167 when being combined to form the first elastic belt 106 and/or second elastic belt 108. In some configurations, mechanical bonding devices may apply mechanical bonds to the at least one of the first substrate 162, second substrate 164, and/or elastic material 167 when being combined to form the first elastic belt 106 and/or second elastic belt 108. Such mechanical bonds may be applied with heat, pressure, and/or ultrasonic devices. In some configurations, mechanical bonding devices may apply bonds that bond the first substrate 162, second substrate 164, and/or elastic material 167 together and/or may act to trap or immobilize discrete lengths of the contracted elastic strands in the first elastic belt 106 and/or second elastic belt 108.

It is to be appreciated that components of the first elastic belt 106 and/or the second elastic belt 108 may be assembled in various ways and various combinations to create various desirable features that may differ along the lateral width and/or longitudinal length of the first elastic belt 106 and/or the second elastic belt 108. Such features may include, for example, Dtex values, bond patterns, aperture arrangements, elastic positioning, Average Dtex values, Average Pre-Strain values, rugosity frequencies, rugosity wavelengths, height values, and/or contact area. It is to be appreciated that differing features may be imparted to various components, such as for example, the first substrate 162, second substrate 164, and elastic material 167 before and/or during stages of assembly of the first elastic belt 106 and/or the second elastic belt 108.

It is to be appreciated that the first elastic belt 106 and/or the second elastic belt 108 may include various configurations of belt elastic materials 167 arranged in relation to each other and to the first substrate 162, and the second substrate 164. As discussed above, the elastic material 167 may include configurations of one or more elastic elements such as strands, ribbons, films, or panels positioned in various arrangements. In some configurations, the elastic material 167 may comprise various elastics, elastic features and arrangements, and processes for assembly, such as described in 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0298586 A1; 2019/0070042 A1; 2018/0168878 A1; 2018/0168877 A1; 2018/0168880 A1; 2018/0170027 A1; 2018/0169964 A1; 2018/0168879 A1; 2018/0170026 A1; 2019/0070041 A1; 2021/0282797 A1; and 2021/0275362 A1, which are all incorporated by reference. It is also to be appreciated that the elastic materials 167 herein may be configured with identical or different colors in various different locations on the first elastic belt 106 and/or the second elastic belt 108.

In some configurations, the elastic material 167 may be configured as elastic strands 168 disposed at a constant interval in the longitudinal direction. In other embodiments, the elastic strands 168 may be disposed at different intervals in the longitudinal direction. In some configurations, the Dtex values of the elastic strands 168 may be constant or varied along the longitudinal direction. In some configurations, the elastic material 167 in a stretched condition may be interposed and joined between uncontracted substrate layers. When the elastic material 167 is relaxed, the elastic material 167 returns to an unstretched condition and contracts the substrate layers. The elastic material 167 may provide a desired variation of contraction force in the area of the ring-like elastic belt. It is to be appreciated that the chassis 102 and elastic belts 106, 108 may be configured in different ways other than as depicted in attached Figures. It is also to be appreciated that the elastic material 167 material may be joined to the substrates continuously or intermittently along the interface between the elastic material 167 material and the substrates. In some configurations, the elastic strands 168 may be in the form of extruded elastic strands, which may also be bonded with the first substrate 162 and/or second substrate 164 in a pre-corrugated configuration, such as disclosed for example in U.S. Pat. No. 5,681,302, which is incorporated by reference herein.

As discussed above for example with reference to FIGS. 3C and 4, the elastic material 167 discussed herein may be in the form of elastic strands 168. In some configurations, the elastic strands 168 may be parallel with each other and/or with the lateral axis 126. It is to be appreciated that the first elastic belt 106 and/or second elastic belt 108 may be configured to include various quantities of elastic strands 168. In some configurations, elastic strands 168 may be grouped in pairs. In some configurations, the first elastic belt 106 and/or second elastic belt 108 may comprise from about 10 to about 1500 elastic strands 168.

It is also to be appreciated that elastic strands 168 herein may comprise various Dtex values, strand spacing values, and pre-strain values and such elastic strands 168 may utilized with other elastic strands to create first and second elastic belts 106, 108 comprising elastic strands 168 in various combinations of Dtex values, strand spacing values, and pre-strain values. For example, in some configurations, the Average-Dtex of one or more elastic strands 168 may be greater than 500. In some configurations, the Average-Dtex of one or more elastic strands 168 may be from about 10 to about 1500, specifically reciting all 1 Dtex increments within the above-recited range and all ranges formed therein or thereby. In some configurations, the elastic strands may have an Average Dtex of from about 200 to about 1000, or from about 300 to about 800, specifically reciting all 1 Dtex increments within the above-recited range and all ranges formed therein or thereby. In some configurations, a plurality of elastic strands 168 may comprise an Average-Strand-Spacing from about 0.25 mm to about 15 mm, specifically reciting all 0.01 mm increments within the above-recited range and all ranges formed therein or thereby. In some configurations, the Average-Pre-Strain of each of a plurality of elastic strands may be from about 50% to about 400%, specifically reciting all 1% increments within the above-recited range and all ranges formed therein or thereby. In some configurations, the Average-Pre-Strain of each of a plurality of elastic strands may be from about 150% to about 300%, or from about 180% to about 300%, or from about 220% to about 275%, specifically reciting all 1% increments within the above-recited range and all ranges formed therein or thereby.

In some configurations, the elastic strands 168 comprise an Average-Strand-Spacing from about 0.25 mm to about 4 mm and an Average-Dtex from about 10 to about 500. In some configurations, the elastic strands 168 may comprise an Average-Strand-Spacing from about 0.25 mm to about 8 mm and an Average-Dtex from about 10 to about 1000. In some configurations, the elastic strands 168 may comprise an Average-Pre-Strain from about 75% to about 300%.

In some configurations, a first plurality of elastic strands may comprise a first Average-Pre-Strain from about 75% to about 300%, and a second plurality of elastic strands may comprise a second Average-Pre-Strain that is greater than first Average-Pre-Strain. In some configurations, a first plurality of elastic strands comprises an Average-Strand-Spacing from about 0.25 mm to about 4 mm and an Average-Dtex from about 10 to about 500; and a second plurality of elastic strands may comprise an Average-Strand-Spacing greater than about 4 mm and an Average-Dtex greater than about 450.

In some configurations, the pitch (measured from the center of one elastic strand to the center of the adjacent elastic strand) between the elastic strands may be from about 4 mm to about 16 mm, or from about 6 mm to about 12 mm, or from about 7 mm to about 9 mm. The pitch between the elastic strands may be substantially equal or may vary along the longitudinal length of the first and/or second belts.

In some configurations, such as shown in FIG. 3C, the elastic strands 168 may be referred to herein as outer waist elastics 170 and inner waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second elastic belt 108. Some elastic strands 168, such as the inner waist elastics 172, may be configured with discontinuities in areas, such as for example, where the first and second elastic belts 106, 108 overlap portions of the chassis 102, such as the absorbent assembly 140. In some configurations, some outer waist elastics 170 and/or inner waist elastics 172 may be configured with discontinuities in areas adapted to refastenably connect with the flanges 300.

As shown in FIG. 3C, the first elastic belt 106 and/or the second elastic belt 108 may be configured with low-stretch zones 701 and high-stretch zones 703. The first elastic belt 106 and/or the second elastic belt 108 may include a first high-stretch zone 703a and a second high-stretch zone 703b separated laterally by a central low-stretch zone 701c. Portions of the chassis 102, such as the backsheet 136 and absorbent assembly 140, may be connected with the first elastic belt 106 and/or the second elastic belt 108 in the central low-stretch zones 701c in the first waist region 116 and/or the second waist region 118. The first elastic belt 106 and/or the second elastic belt 108 may also include a first lateral low-stretch zone 701a and a second lateral low-stretch zone 701b. In some configurations, the second belt 108 may include first and second lateral low-stretch zones 701a, 701b located in areas where flanges 300 may be refastenably connected with the second belt 108. In some configurations, the first and second low-stretch zones 701a, 701b may be positioned adjacent to the outer laterally extending edge 109a of the second belt 108. The first and second low-stretch zones 701a, 701b may have a lateral width of from about 20 mm to about 50 mm, or from about 30 mm to about 40 mm. The first and second low-stretch zones 701a, 701b may have a longitudinal length of from about 110 mm to about 160 mm, or from about 115 mm to about 155 mm, or from about 120 mm to about 150 mm. In some configurations, the longitudinal length of the first and/or second low-stretch zones 701a, 701b may be about 10 mm longer than the longitudinal length of the fastener component 304.

In some configurations, the first and/or second low-stretch zones 701a, 701b of the second belt 108 may comprise a stiffening patch 400 in areas where flanges 300 may be refastenably connected with the second belt 108. Without being limited by theory, it is believed that the stiffening patch 400 may help to increase the stiffness of the first and second end regions of the second belt and aid in fastening (and refastening) of the belts by reducing the collapse of the second belt and/or creating a flatter area that can engage more effectively with the fastener component on the first belt.

The high-stretch zones 703 are elasticated by the elastic material 167, such as the elastic strands 168, 172; and the low-stretch zones 701 may comprise cut lines separating the elastic material 167, such as the elastic strands 168, 172. In some configurations, the elastic material 167 may be cut in an unbonded region where the elastic material is not bonded with first substrate 162 and the second substrate 164. Thus, the elastic material 167 retracts from the unbonded region and form low-stretch zone 701. In some configurations, the elastic material 167 may be cut into several discrete pieces. In turn, the low-stretch zones 701 define regions of the first elastic belt 106 and/or the second elastic belt 108 that have relatively less elasticity than the high-stretch zones 703. The discrete elastic material 167 that has been cut and which are elastically contracted do not add any substantial amount of elastication to the low-stretch zone 701. As such, upon application of a force, the high-stretch zones 703 will elongate more than the low-stretch zones 701. As provided above, the terms “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force. In some configurations, the first elastic belt 106 and/or the second elastic belt 108 may be configured with high-stretch zones 703 that are elastic and may be configured with low-stretch zones 701 that are not elastic or “inelastic.”

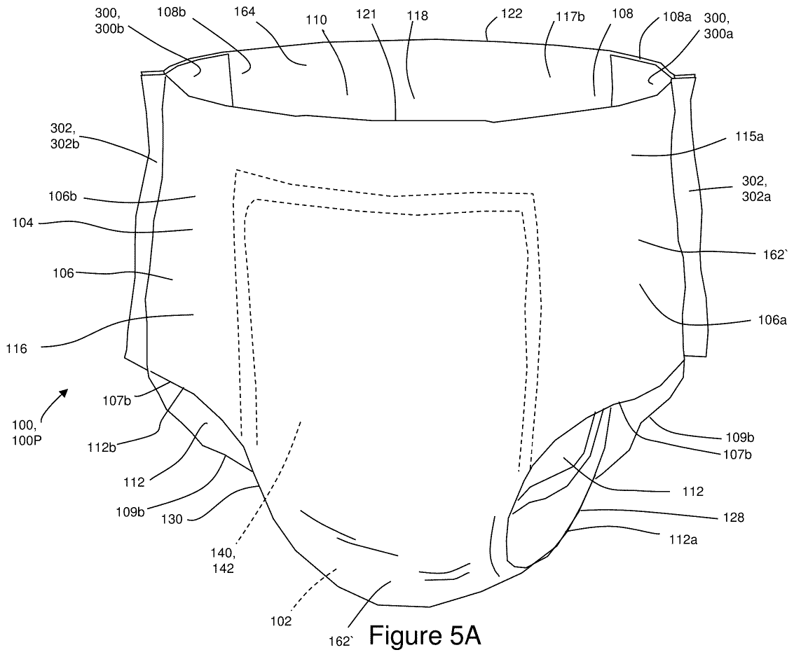

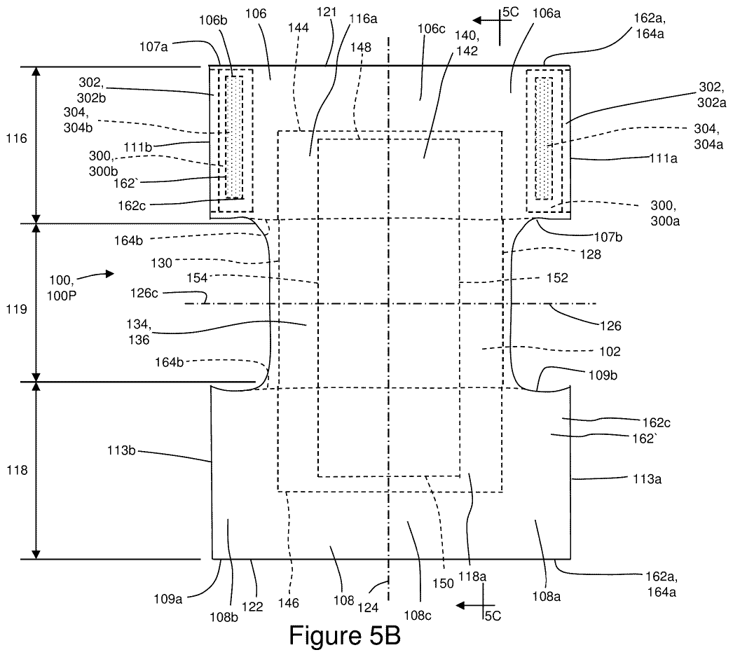

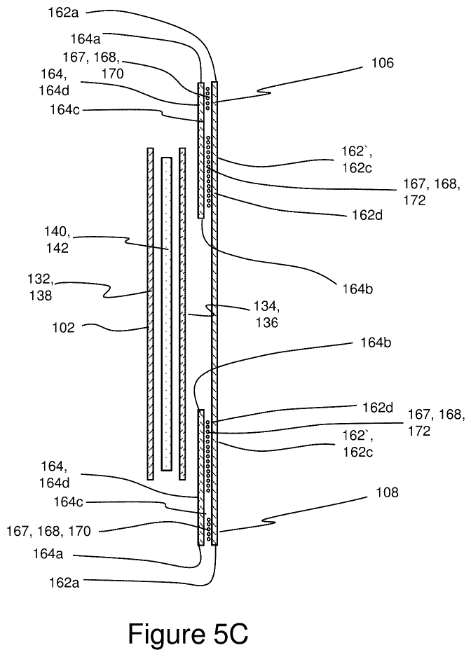

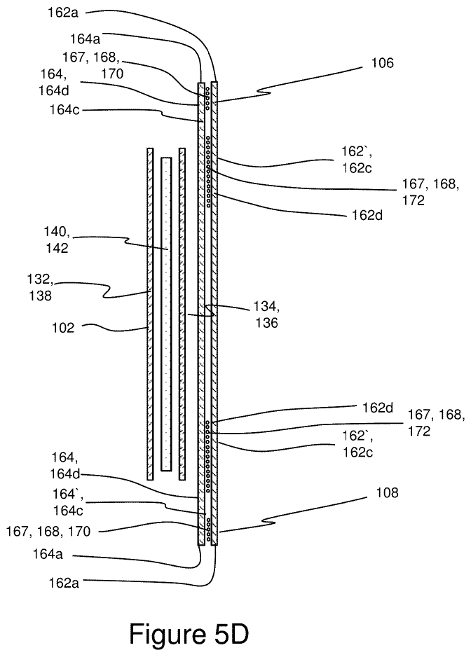

It is also to be appreciated diaper pants 100P may be configured with the first substrate 162 and/or the second substrate 164 that may extend continuously from the first belt 106 to the second belt 108. For example, the first substrate 162 may be configured to define a continuous outer cover 162′ that extends contiguously from the first waist edge 121 to the second waist edge 122, such as shown in FIGS. 5A-5C. FIG. 5D shows a diaper pant 100P with both the first substrate 162 configured to define a continuous outer cover 162′ and the second substrate 164 configured to define a continuous inner layer 164′ that extend contiguously from the first waist edge 121 to the second waist edge 122. It is also to be appreciated that diaper pants 100P with continuous outer covers, such as shown in FIGS. 5A-5D may also be configured to include various aspects of the elastic material 167, flanges 300, and fastener components 304 discussed herein as well as waist edge configurations described about with reference to FIGS. 4 through 4A2.

As discussed above, the diaper pant 100P may include flanges 300 bonded with opposing end regions of the first belt 106, and the flanges 300 may be refastenably connected with opposing end regions of the second belt 108. As shown in FIGS. 2A and 2B, the flanges 300 may each include a first surface 308 and an opposing second surface 310. The flanges 300 may further comprise a first lateral end region 312 and a second lateral end region 314. The first surface 308 of the first lateral end region 312 of the first flange 300a may be bonded with the first end region 106a of the first belt 106 at the first flange seam 302a, and the second surface 310 of the second lateral end region 314 of the first flange 300a may be refastenably connected with the first end region 108a of the second belt 108. In some configurations, a first fastener component 304a on the second surface 310 of the second lateral end region 314 of the first flange 300a may be refastenably connected with first end region 108a of the second belt 108. In addition, the first surface 308 of the first lateral end region 312 of the second flange 300b may be bonded with the second end region 106b of the first belt 106 at the second flange seam 302b, and the second surface 310 of the second lateral end region 314 of the second flange 302b may be refastenably connected with the second end region 108b of the second belt 108. In some configurations, a second fastener component 304b on the second surface 310 of the second lateral end region 314 of the second flange 300a may be refastenably connected with second end region 108b of the second belt 108. In some configurations, the fastener component 304 may be positioned laterally inboard of the flange seam 302. In some configurations, the fastener component 304 and the flange seam 302 may be spaced apart by a distance of from about 3 mm to about 11 mm, or from about 5 mm to about 8 mm.

With reference to FIG. 2B, when the diaper pant 100P is in a compressed, folded state, such as when placed in a package, with the wearer facing surface 115b of the first belt 106 placed in a direct facing relationship with the wearer facing surface 117b of the second belt 108, the first surfaces 308 of the first flange 300a and the second flange 300b are in a direct facing relationship with the wearer facing surface 115b of the first belt 106. In addition, the second surfaces 310 of the first flange 300a and the second flange 300b are in a direct facing relationship with the wearer facing surface 117b of the second belt 108. As shown in FIG. 2A, when the diaper pant 100P is in an expanded state, such as when being worn or being placed on a wearer, with the wearer facing surface 115b of the first belt 106 separated from the wearer facing surface 117b of the second belt 108, the first surfaces 308 of the first flange 300a and the second flange 300b define wearer facing surfaces and may be in a direct facing relationship with a wearer. In addition, the second surfaces 310 of the first flange 300a and the second flange 300b define garment facing surfaces and are maintained in a direct facing relationship with the wearer facing surface 117a of the second belt 108.

As discussed herein and as illustrated in the accompanying figures, it is to be appreciated that the first belt 106 and/or second belt 108 may be configured as laminates that may comprise regions having different numbers of layers of substrates. As such, it is to be appreciated that flanges may be bonded with and refastenably connected with various arrangements of layers of substrates of the first belt 106 and/or the second belt 108.

For example, FIG. 2C1 is a cross sectional view of first and second belts 106, 108 configured with laminate structures that correspond with laminate structures shown and described above with reference to FIG. 4. As shown in FIG. 2C1, the first surface 308 of the flange 300 may be bonded with the wearer facing surface 115b of the first belt 106, and the second surface 310 of the flange 300 may be refastenably connected with the wearer facing surface 117b of the second belt 108. More particularly, the first surface 308 of the flange 300 may be bonded with the second substrate 164 of the first belt 106, and a fastener component 304 on the second surface 310 of the flange 300 may be refastenably connected with the second substrate 164 of the second belt 108.

In other configurations described above, at least one substrate of a first belt 106 and/or a second belt 108 may be folded to partially overlap itself and/or another substrate, which in turn may define regions of the belt having different numbers of layers of substrates. For example, FIG. 2C2 is a cross sectional view of first and second belts 106, 108 configured with laminate structures that correspond with laminate structures shown and described above with reference to FIGS. 4A, 4B, and 4A1. As shown in FIG. 2C2, the first surface 308 of the flange 300 may be bonded with the second substrate 164 as well as the folded portion 162g of the first substrate 162 of the first belt 106. In addition, a fastener component 304 on the second surface 310 of the flange 300 may be refastenably connected with the second substrate 164 as well as the folded portion 162g of the first substrate 162 of the second belt 108.

FIG. 2C3 shows yet another cross sectional view of first and second belts 106, 108 configured with laminate structures that correspond with laminate structures shown and described above with reference to FIG. 4A2. As shown in FIG. 2C3, the first surface 308 of the flange 300 may be bonded with the second substrate 164 in regions where the second substrate 164 overlaps the folded portion 162g of the first substrate 162 of the first belt 106. In addition, a fastener component 304 on the second surface 310 of the flange 300 may be refastenably connected with the second substrate 164 in regions where the second substrate 164 overlaps the folded portion 162g of the first substrate 162 of the second belt 108.

In other configurations, second belt 108 may comprise a stiffening patch 400. FIG. 2C4 shows a cross sectional view of first and second belts 106, 108 configured with laminate structures that correspond with laminate structures shown and described above with reference to FIGS. 4A, 4B, and 4A1, wherein second belt 108 comprises a stiffening patch 400. As shown in FIG. 2C4, the stiffening patch 400 may comprise a first surface 401 and a second surface 403, wherein the second surface 403 may be bonded with a portion of the second substrate 164 of the second belt 108 and a portion of the folded portion 162g of the first substrate 162 of the second belt 108. In addition, a fastener component 304 on the second surface 310 of the flange 300 may be refastenably connected with the first surface 401 of the stiffening patch 400.

FIG. 2C5 shows yet another cross sectional view of first and second belts 106, 108 configured with laminate structures that correspond with laminate structures shown and described above with reference to FIGS. 4A, 4B, and 4A1, wherein second belt 108 comprises a stiffening patch 400. As shown in FIG. 2C5, the stiffening patch 400 may be positioned between the first substrate 162 and the second substrate 164 of the second belt 108. In addition, a fastener component 304 on the second surface 310 of the flange 300 may be refastenably connected with the second substrate 164 as well as the folded portion 162g of the first substrate 162 of the second belt 108.

FIG. 2C6 shows yet another cross sectional view of first and second belts 106, 108 configured with laminate structures that correspond with laminate structures shown and described above with reference to FIGS. 4A, 4B, and 4A1, wherein second belt 108 comprises a stiffening patch 400. As shown in FIG. 2C6, the second surface 403 of the stiffening patch 400 may be bonded with the second substrate 164 of the second belt 108 and the folded portion 162g of the first substrate 162 of the second belt may overlap and be bonded with a portion of the first surface 401 of the stiffening patch 400.

Stiffening patches 400 may be connected with the belt in various ways, such as mechanical bonding, adhesive bonding, or both. While FIGS. 2C4-2C6 illustrate stiffening patch 400 positioned on second belt 108, it is to be appreciated that in some configurations where flanges 300 may be bonded with the opposing end regions of the second belt, the stiffening patches may be positioned on the first belt 106.

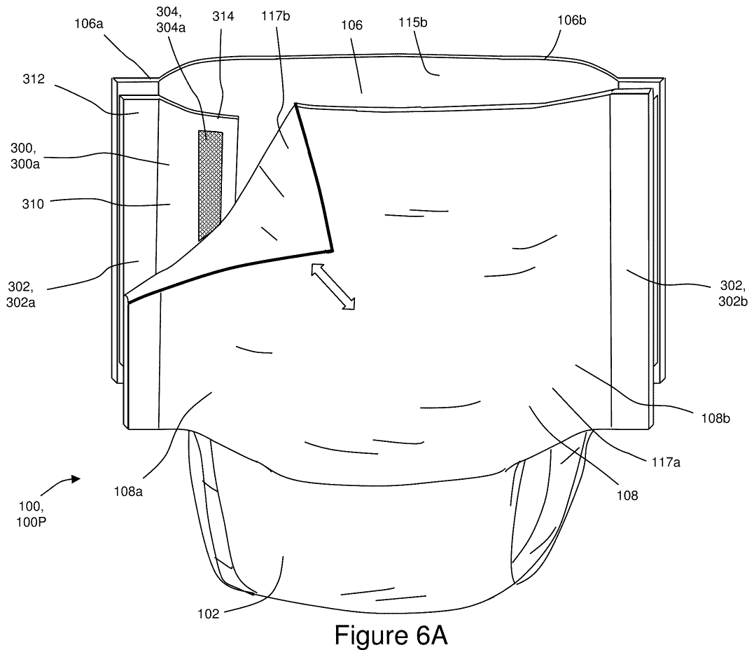

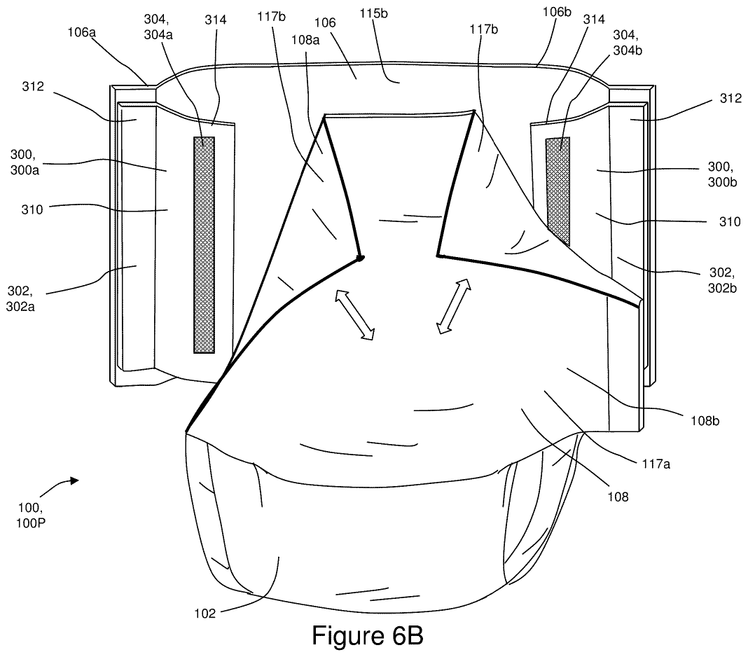

As discussed above, the diaper pants 100P described herein may include one or more refastenable connections between the first belt 106 and the second belt 108. For example, FIGS. 1, 6A, and 6B show an example diaper pant 100P with a first belt 106 refastenably connected with the second belt 108. The refastenable connections between the belts may allow the first elastic belt 106 and the second belt 108 to be partially or completely separated from each other, such as when inspecting a diaper pant 100P while being worn in order to determine if the diaper pant 100P may be soiled. If it is determined that the diaper pant 100P is suitable for continued use, the first and second belts 106, 108 may be reconnected with each other. For example, FIG. 6A is a perspective view of the diaper pant 100P of FIG. 1 showing the first end region 108a of the second belt 108 partially disconnected from the first flange 300a. From the configuration shown in FIG. 6A, it is to be appreciated that the disconnected portion of the first end region 108a of the second belt 108 may be reconnected with the first fastener component 304a with the application of forces to the second belt 108 generically represented by a bi-directional arrow. It is also to be appreciated that the remaining connected portion of the first end region 108a of the second belt 108 may be further disconnected from the first fastener component 304a with the application of forces to the second belt 108 generically represented by a bi-directional arrow.

FIG. 6B is a perspective view of the diaper pant of FIG. 6A showing the first end region 108a of the second belt 108 having been completely disconnected from the first fastener component 304a on the first flange 300a. FIG. 6B also shows the second end region 108b of the second belt 108 partially disconnected from a second flange 300b. From the configuration shown in FIG. 6B, it is to be appreciated that the disconnected portions of the first end region 108a and the second end region 108b of the second belt 108 may be reconnected with the first and second fastener components 304a, 304b, respectively, with the application of forces to the second belt 108 generically represented by the bi-directional arrows. It is also to be appreciated that the remaining connected portion of the second end region 108b of the second belt 108 may be further disconnected from the second fastener component 304b with the application of forces to the second belt 108 generically represented by a bi-directional arrow.

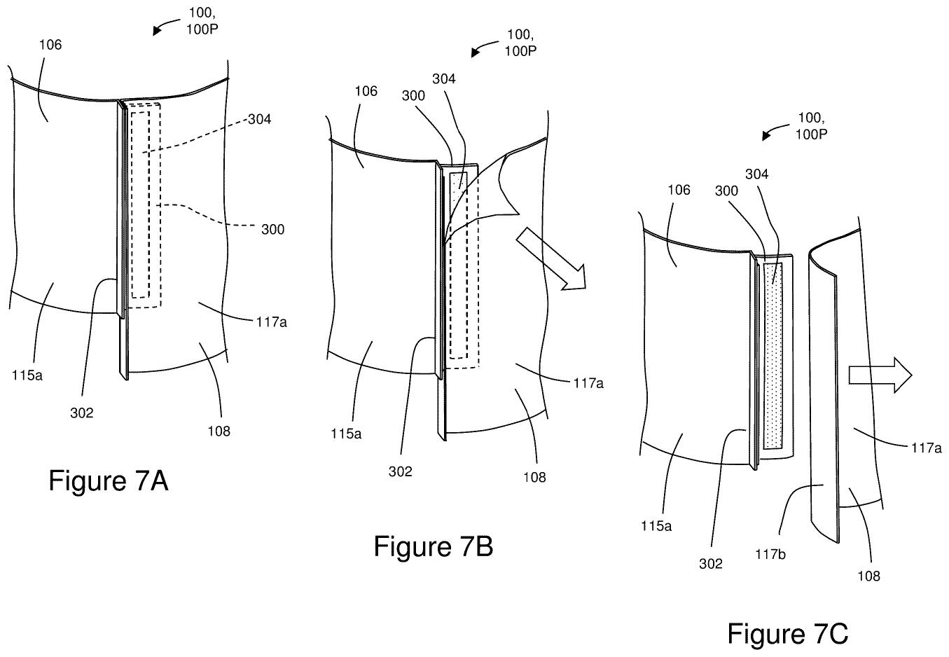

As previously discussed, the refastenable connections between the belts 106, 108 may be configured to allow the first elastic belt 106 and the second elastic belt 108 to be relatively easily and completely separated from each other, such as when removing the diaper pant 100P from a wearer. For example, FIGS. 7A-7C illustrate a progression whereby the first belt 106 and the second belt 108 may be completely disconnected from each other. In particular, FIG. 7A is a detailed view of a diaper pant 100P showing a flange 300 bonded with a first belt 106 and refastenably connected with a second belt 108. FIG. 7B is a detailed view of the diaper pant 100P of FIG. 7A showing the second belt 108 partially disconnected from the flange 300, and FIG. 7C is a detailed view of the diaper pant 100P of FIG. 7B showing the second belt 108 completely disconnected from the flange 300. As such, once the opposing end portions of the first elastic belt 106 and the second elastic belt 108 are completely disconnected, the diaper pant 100P may be removed from a wearer in a similar fashion to a taped diaper.

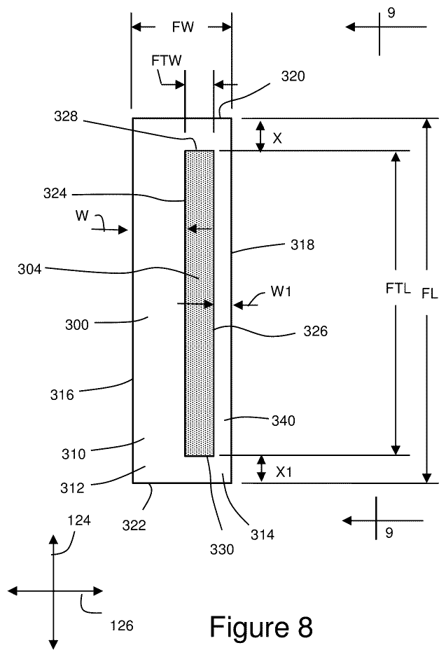

It is to be appreciated that the flange 300 and/or the fastener component 304 may comprise any of a wide variety of shapes, including rectangles or other polygons, circles, ovals, shapes having exterior convexities or concavities or combinations thereof, or one or a plurality of lines or geometric shapes forming an array. It is also to be appreciated that more than one fastener component 304 may be positioned on a flange. It is also to be appreciated that the fastener component 304 and the flange 300 may be configured with various sizes and shapes. For example, FIG. 8 illustrates a detailed planar view of a fastener component 304 positioned on the second surface 310 of a flange 300. To provide a frame of reference, a longitudinal axis 124 and a lateral axis 126 are illustrated in FIG. 8 and correspond with the directional frames of reference provided by the longitudinal axis 124 and lateral axis 126, respectively, shown in other figures herein. As shown in FIG. 8, the flange 300 may define a long lateral width FW and longitudinal length FL, and the fastener component 304 may define a lateral width FTW and a longitudinal length FTL. In some configurations, FW may be equal to or greater than FTW, and FL may be equal to or greater than FTL. The flange 300 may have a lateral width FW of from about 26 mm to about 40 mm, or from about 30 mm to about 38 mm, and a longitudinal length FL of from about 110 mm to about 130 mm, or from about 115 mm to about 125 mm. The fastener component 304 may have a lateral width FTW of from about 10 mm to about 18 mm, or from about 12 mm to about 15 mm, and a longitudinal length FTL of from about 105 mm to about 120 mm, or from about 108 mm to about 115 mm. In some configurations, gap regions 340 may be defined on the flange 300 by the absence of portions of the fastener component 304.

With continued reference to FIG. 8, the flange 300 may comprise a first side edge 316 laterally separated from a second side edge 318 and may comprise a first end edge 320 longitudinally separated from a second end edge 322. In addition, the fastener component 304 may comprise a first side edge 324 laterally separated from a second side edge 326 and may comprise a first end edge 328 longitudinally separated from a second end edge 330. In some configurations, the first side edge 324 of fastener component 304 may be coterminous with or laterally inboard from the first side edge 316 of the flange 300, and/or the second side edge 326 of the fastener component 304 may be coterminous with or laterally inboard from the second side edge 318 of the flange 300. In some configurations, the first side edge 324 of fastener component 304 may be positioned laterally inboard from the first side edge 316 of flange 300 a distance W of from about 8 mm to about 21 mm, or from about 10 mm to about 18 mm, and/or the second side edge 326 of fastener component 304 may be positioned laterally inboard from the second side edge 318 of flange 300 by a distance W1 of from about 1 mm to about 9 mm, or from about 3 mm to about 7 mm. Without being limited by theory, it is believed that if the fastener component 304 is positioned greater than about 9 mm from the second side edge 318 of flange 300, gap region 340 of flange 300 may fold over and cover a portion of fastener component 304, thus interfering with the engagement of fastener component 304 with the opposing belt.

In some configurations, the first end edge 328 of the fastener component 304 may be coterminous with or longitudinally inboard from first end edge 320 of the flange 300, and/or the second end edge 330 of the fastener component 304 may be coterminous with or longitudinally inboard from second end edge 322 of the flange 300. In some configurations, the first end edge 328 of fastener component 304 may be positioned longitudinally inboard from first end edge 320 of flange 300 by a distance X of from about 2 mm to about 8 mm, or from about 4 mm to about 6 mm, and/or the second end edge 330 of fastener component 304 may be positioned longitudinally inboard from second end edge 322 of flange 300 by a distance X1 of from about 2 mm to about 8 mm, or from about 4 mm to about 6 mm. Without being limited by theory, it is believed that such placement of the first end edge 328 and/or second end edge 330 of fastener component 304 with respect to the first and/or second end edges 320, 330 of flange 300 may help to avoid the fastener component 304 from protruding longitudinally outboard of the first waist edge and coming into contact with the skin during use.

It is also to be appreciated that the fastener component may be configured in various ways and may be connected with the first waist region in various ways. As discussed above, the fastener component may comprise a hook material that can refastenably engage with substrates, such as nonwovens for example. For example, the fastener component may comprise a base or laminate structure comprising protrusions, such as hooks, wherein the base or laminate structure is bonded with a structure of the first waist region, such as a flange or a belt, which may comprise a nonwoven. It is to be appreciated that the base or laminate structure may be bonded with the first waist region in various ways, such as for example, with mechanical bonds, thermal bonds, ultrasonic bonds, and/or adhesive bonds or combinations thereof.

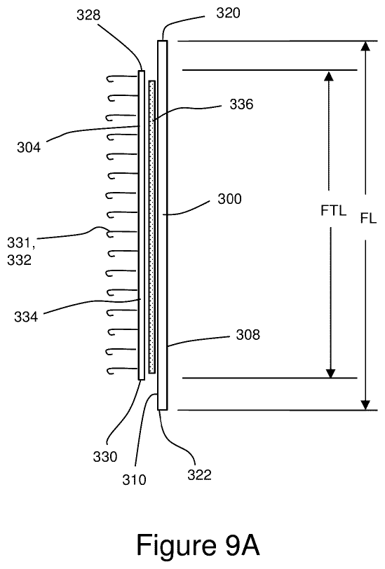

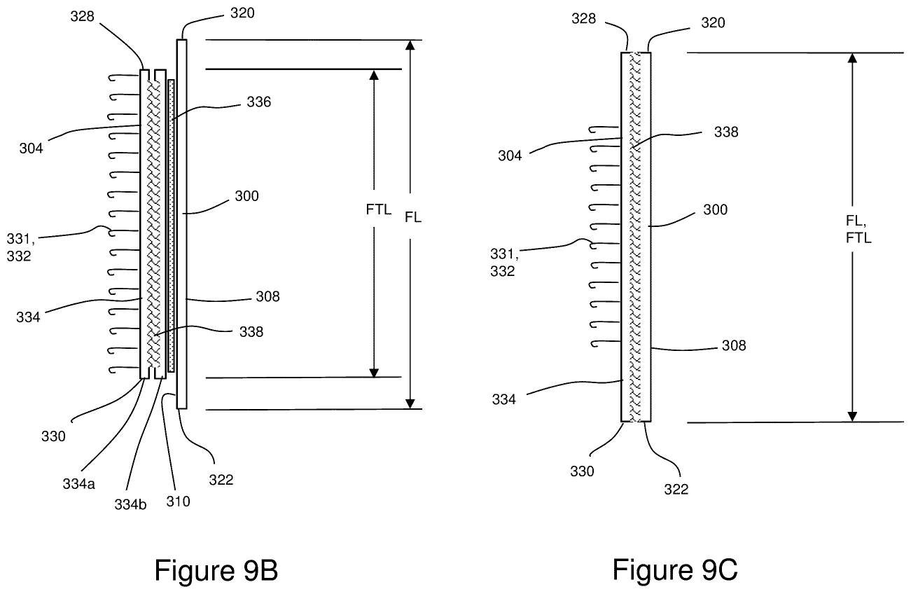

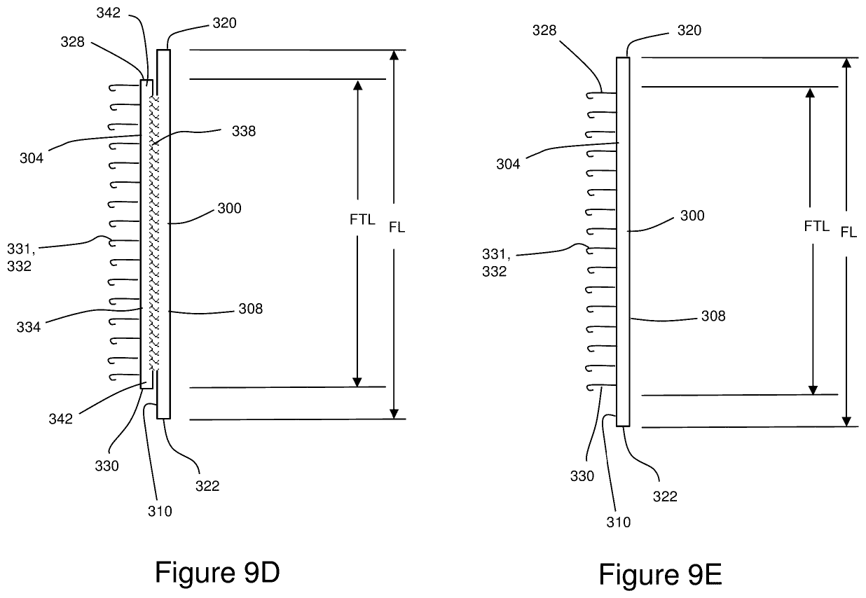

For example, as shown in FIG. 9A, the fastener component 304 may comprise a plurality of protrusions 331 extending from a base 334. Protrusions 331 may be hooks, nubs, or other protrusions having various shapes configured to engage a receiving component, another fastener component, and/or landing zone component. In some configurations, the hooks may be any shape, such as nub, “mushroom”, “J” shape, “T” shape, hook shape, or any other shape known in the art, so long as the hooks can refastenably engage a receiving component, another fastener component, and/or landing zone component. Additional details of hook shapes, and tools and methods for forming such hooks, are disclosed in U.S. Pat. No. 8,784,722. For example, FIGS. 2-2N of U.S. Pat. No. 8,784,722 depicts a plurality of hook shapes.

In some configurations, the fastener component 304 may comprise hooks 332 protruding from a base 334, and adhesive 336 may connect the base 334 of the fastener component 304 with the second surface 310 of the flange 300. It is to be appreciated that the fastener component 304 may be connected with flange 300 by mechanical bonding in addition to or instead of adhesive 336. It is also to be appreciated that the base 334 may be configured in various ways. For example, the base 334 may comprise a thermoplastic film. In addition, the adhesive 336 between the base 334 and the flange 300 may extend longitudinally for the entire length FTL or less than the entire length FTL of the base. Further, the adhesive 336 between the base 334 and the flange 300 may extend laterally for the entire width FTW or less than the entire length FTW of the base.

In some configurations, the base 334 may comprise a laminate with various layers bonded together. It is also to be appreciated that such layers may be bonded together in various ways, such as with adhesive, mechanical bonding, and/or extrusion bonding. In some configurations, layers of the base 334 may be bonded together with extrusion or melt type bonding such as disclosed for example in U.S. Patent Publication No. 2021/0045931A1. For example, as shown in FIG. 9B, the base 334 may comprise a thermoplastic film layer 334a and a nonwoven backing layer 334b, wherein the thermoplastic film layer 334a is bonded with the nonwoven backing layer 334b with extrusion bonds 338. In turn, the nonwoven backing layer 334b may be bonded with the flange 300 with adhesive 336.

In some configurations, such as shown in FIGS. 9C and 9D for example, the base may be bonded directly with the flange with extrusion or melt type bonding, such as disclosed for example in U.S. Patent Publication No. 2021/0045931A1. For example, the base 334 may comprise a thermoplastic film layer and the flange 300 may comprise a nonwoven layer, wherein the thermoplastic film base 334 is bonded with the nonwoven flange 300 with extrusion bonds 338. In some configurations, such as shown in FIG. 9C, the base 334 may extend longitudinally for the entire length FL of the flange 300, such as from the first end edge 320 of the flange 300 to the second end edge 322 of the flange 300, and the hooks 332 may extend longitudinally for less than the entire length FL of the flange 300. In addition, the extrusion bonds 338 between the base 334 and the flange 300 may extend longitudinally for the entire lengths FL, FTL of the flange 300 and base 334. Further, the extrusion bonds 338 between the base 334 and the flange 300 may extend laterally for the entire width FTW or less than the entire length FTW of the base 334. In some configurations, such as shown in FIG. 9D, the base 334 may extend longitudinally for a length FTL that is less than the entire length FL of the flange 300. In addition, the extrusion bonds 338 between the base 334 and the flange 300 may extend longitudinally for the entire length FTL or less than the entire length FTL of the base 334. Further, the extrusion bonds 338 between the base 334 and the flange 300 may extend laterally for the entire width FTW or less than the entire length FTW of the base 334. As such, in some configurations, edge regions 342 of the base 334 adjacent the first side edge 324, the second side edge 326, the first end edge 328, and/or the second end edge 330 may not be bonded with the flange 300.

In some configurations, protrusions 331 may be integrally formed from the flange 300. In some configurations, protrusions 331 may be hooks 332 integrally formed from the flange 300, which may for example be in the form of a nonwoven as disclosed in, for example, U.S. Pat. No. 8,784,722. For example, as shown in FIG. 9E, the fastener component 304 may be integrally formed from materials of the flange 300 or may be integrally formed from other materials that may then be attached with the flange 300.

It is to be appreciated that the discussions and descriptions above with regard to bonding arrangements and configurations between the fastener components 304 and the flange 300 are also applicable to bonding arrangements and configurations between the fastener components 304 and materials of the first and/or second waist region, such as first and/or second belts 106, 108.

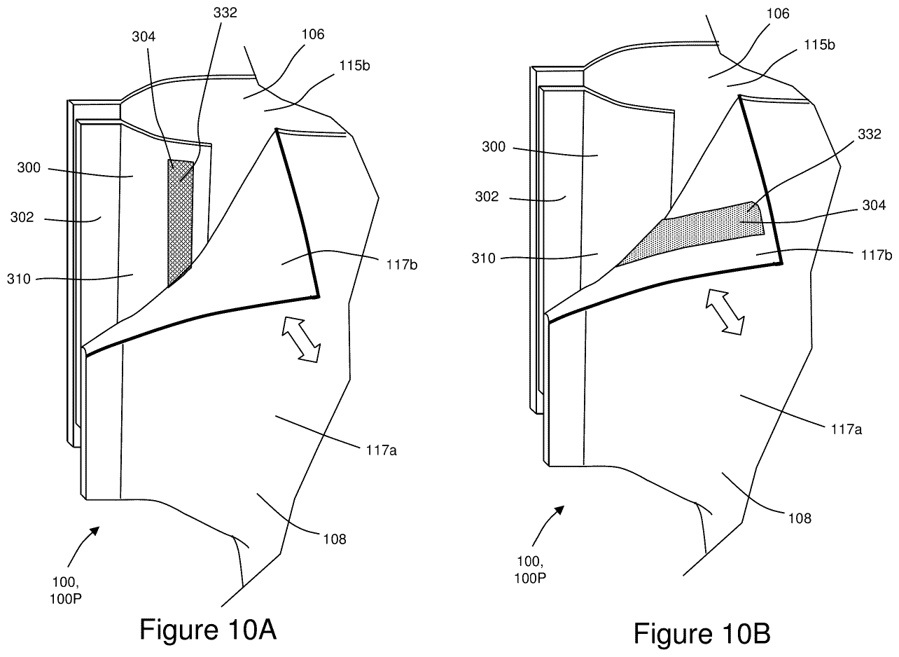

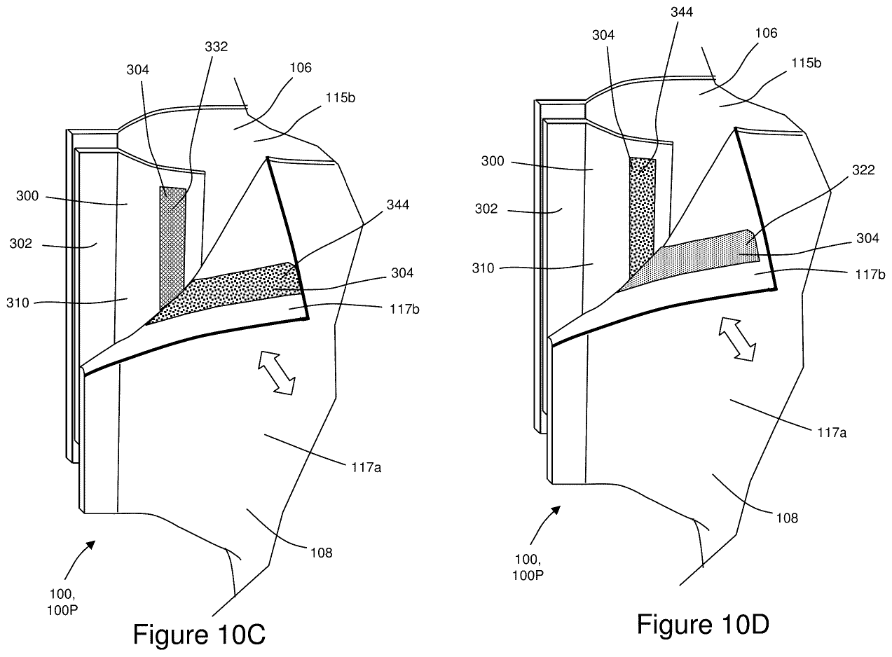

As discussed above, it is to be appreciated that the various arrangements and types of fastener components 304 and flanges 300 may be configured to refastenably connect opposing end regions of the first belt 106 with the second belt 108. For example, FIG. 10A is a detailed view of a diaper pant 100P showing a second belt 108 directly refastenably connected with a fastener component 304 on a flange 300. The fastener component 304 may comprise hooks 332 adapted to directly refastenably connect with material defining the wearer facing surface 117b of the second belt 108, such as for example the first substrate 162 and/or 164 of the second belt 108. In another example, FIG. 10B shows a detailed view of a diaper pant 100P with a fastener component 304 comprising hooks 332 on a wearer facing surface 117b of the second belt 108 that may be adapted to directly refastenably connect with material defining the second surface 310 of the flange 300. In yet another example, FIG. 10C shows a detailed view of a diaper pant 100P with a fastener component 304 comprising hooks 332 on the second surface 310 of the flange 300 and a fastener component 304 comprising loops 344 on the wearer facing surface 117b of the second belt 108, wherein the hooks 332 are adapted to refastenably connect with the loops 344. In still another example, FIG. 10D a detailed view of a diaper pant 100P with a fastener component 304 comprising loops 344 on the second surface 310 of the flange 300 and a fastener component 304 comprising hooks 332 on the wearer facing surface 117b of the second belt 108, wherein the hooks 332 are adapted to refastenably connect with the loops 344. It is to be appreciated that in some configurations, adhesive may be used in place of or in addition to the hooks as described above with reference to FIGS. 10A-10D.

In some configurations, the fastener component 304 may comprise from about 1300 to about 2300 hooks per square inch, as seen under high resolution image, or from about 1500 to about 2200 hooks per square inch, or from about 1700 to about 2000 hooks per square inch. The fastener component 304 may comprise hooks having a basis weight (without adhesive) of from about 70 to about 100 g/m², or from about 75 to about 95 g/m², or from about 80 to about 90 g/m².

In some aspects, the fastener component 304 and/or the hooks may comprise a material selected from the group consisting of polypropylene, polylactic acid, polyolefin copolymers, polyolefin blends, and combinations thereof. Suitable examples of fastener components may include commercially available hooks from Aplix SA France under the tradename SoftGrip® and/or SoftGrip® Gen2, and from 3M USA under tradename CHK07197. Depending on the engaging substrate, the hook type may be changed to better suit engagement. Still other hook materials are described in U.S. Pat. No. 5,058,247, which is incorporated by reference.

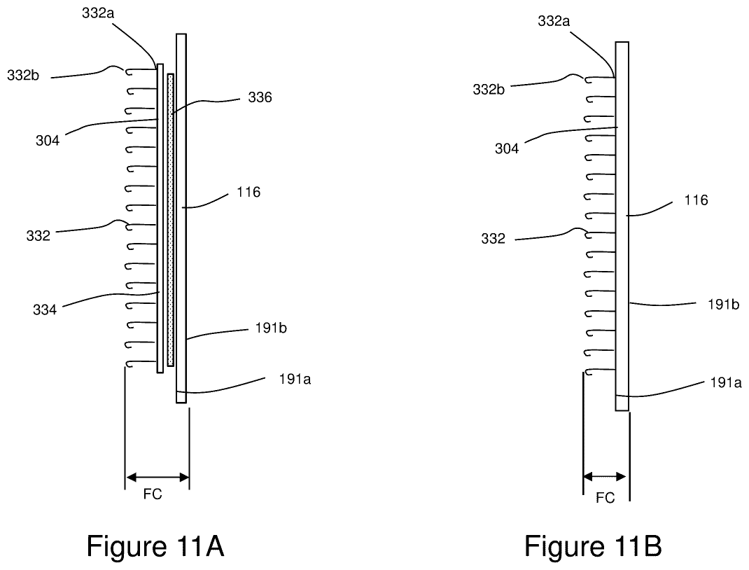

FIGS. 11A and 11B are cross-section views of the fastener component 304 positioned on first waist region 116. In some configurations, fastener component 304 may comprise a plurality of hooks 332 extending from a proximal end 332a to a distal end 332b. The first waist region 116 may comprise a total fastener caliper, FC, measured from the second surface 191b of the first waist region 116 to a distal end 332b of the hooks 332. In some configurations, the total fastener caliper may be from about 0.3 mm to about 1.2 mm, or from 0.3 mm to about 1.0 mm, or from about 0.3 mm to about 0.7, mm as measured according to the Caliper Test Method. In some configurations, as shown in FIG. 11A, the fastener component 304 may comprise a plurality of hooks 332 protruding from the base 334, and adhesive 336 may connect the base 334 of the fastener component 304 with the first and/or second end regions 216a, 216b of the first waist region 116. As discussed above, the base may be bonded to the flange. In some configurations, as shown in FIG. 11B, hooks 332 may be integrally formed from materials of the first waist region 116, such as a flange.

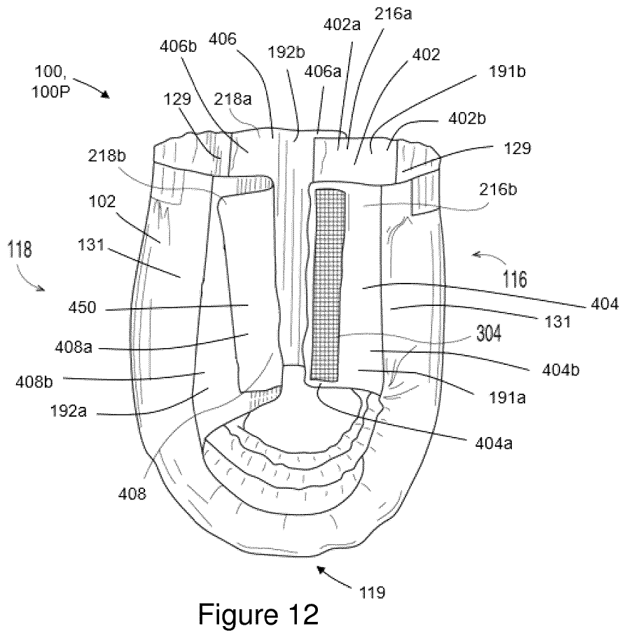

FIG. 12 illustrates an additional example of a diaper pant 100P comprising refastenable side panels. In some configurations, a first portion of the first waist region 116 may extend laterally outward from a first chassis side region 129 forming a first side panel 402, and a second portion of the first waist region 116 may extend laterally outward from a second chassis side region 131 forming a second side panel 404. The first side panel 402 may comprise a first end region 402a and a second end region 402b opposite the first end region 402a. The second side panel 404 may comprise a first end region 404a and a second end region 404b opposite the first end region 404a. A first fastener component 304 may be disposed on the first end region 402a of the first side panel 402 and a second fastener component 304 may be disposed on a first end region 404a of the second side panel 404. In some configurations, a first portion of the second waist region 118 may extend laterally outward from a first chassis side region 129 forming a third side panel 406, and a second portion of the second waist region 118 may extend laterally outward from a second chassis side region 131 forming a fourth side panel 408. The third side panel 406 may comprise a first end region 406a and a second end region 406b opposite the first end region 406a. The fourth side panel 408 may comprise a first end region 408a and a second end region 408b opposite the first end region 408a. The first end regions 406a, 408a of the third and fourth side panels 406, 408 may each comprise a landing zone 450 wherein the fastener components may refastenably engage. It is to be appreciated that the discussions and descriptions above with regard to the fastener components disposed on the first and second side panels are also applicable to configurations where the fastener components are disposed on the third and fourth side panels.

The side panels, or portions thereof, may be elastic or may have elastic panels. In some configurations, the side panel may comprise an elastic film or elastic strands positioned intermediate a first nonwoven material and a second nonwoven material. The elastic film may or may not be apertured. The side panels may be integral (e.g., extension of the outer cover material, the backsheet, and/or the topsheet) or may be discrete components attached to the chassis on a wearer-facing surface, on the garment-facing surface, or intermediate the two surfaces.

It is to be appreciated that the discussions and descriptions above with regard to bonding arrangements and configurations between the fastener components 304 and the flange 300 are also applicable to bonding arrangements and configurations between the fastener components 304 and materials of the side panels.

In some configurations, the fastener component may comprise a Peak Peel Strength of about 2.0 N/inch or more, or from about 2.0 N/inch to about 10.0 N/inch, or from about 2.0 N/inch to about 5.0 N/inch, as measured according to the Fastener Shear and Peel Strength Test Method.

The first and second end regions of the first waist region may exhibit an Average Peak Stress of from about 0.10 to about 1.0 N/cm, measured at a position within the fastener component according to the Fastener Ultra Sensitive 3 Point Bending Method.

When in a fastened configuration, the absorbent article may pass the Whole Article Force Test.

The first belt 106, second belt 108, and/or flange 300 may comprise one or more design elements adapted to indicate the proper alignment of the first belt 106, second belt 108, and/or flange 300 to create a ring-like elastic belt 104 including one or more of insignia, letters, words, graphics, logos, colors, fonts, shapes, or combinations thereof. In some configurations, the first and second belts 106, 108 may have a first color and the flange 300 may comprise a second color that is different from the first color. When the first belt and second belts 106, 108 are connected, the second color of the flange 300 may be substantially covered by the belts, signaling to the caregiver that the belts are properly connected and provide confidence that the absorbent article is properly fitted to the user. In other configurations, the first belt 106 may comprise a first portion of a graphic and the second belt may comprise a second portion of the graphic When the first and second belts are connected, the graphic may be completed, signaling to the caregiver that the belts are properly connected and provide confidence that the absorbent article is properly fitted to the user.

The one or more design elements may be printed on the garment facing surface of the first belt 106, second belt 108, and/or flanges 300. The design elements may extend substantially around the entire circumference of the diaper pant 100P and may be disposed across longitudinal side edges of the first and second belts 106, 108 in the manner described in U.S. Pat. No. 9,498,389 to create a more underwear-like article.

### EXAMPLES

The following data and examples, including comparative examples, are provided to help illustrate the absorbent articles described herein. The exemplified structures are given solely for the purpose of illustration and are not to be construed as limitations of the present disclosure, as many variations thereof are possible without departing from the spirit and scope of the invention.

A series of measurements were done on fastener components (Hooks #1-3) and landing zone materials (Landing Zones #1-5) as described below. The fastener components comprise hooks extending from a base and are bonded to a nonwoven flange (Flange #1 described below).

### Nonwoven Flange:

FLANGE #1: Flange #1 is a 40 gsm nonwoven that is available from Berry USA under tradename Berry: MONC-40-YYY-SS-WHITE-PF. The nonwoven is made of polypropylene and has SS structure, i.e., has two layers spunbond, spunbond respectively. The nonwoven is thermally bonded and has diamond or oval shape bond pattern.

### Fastener Components:

HOOK #1: Hook #1 includes an 80 gsm hook (no adhesive coating on the backing) that is available from 3M USA under the tradename CHK07197. The hooks are described by 3M as micro-replicated round-capped mushroom hooks, comprised of polypropylene, and have approximately 2,000 hooks per square inch. Hook #1 is bonded to Flange #1 via 60 gsm of H4376 adhesive available from Bostik (Colombes, France).

HOOK #2: Hook #2 includes an 82 gsm hook that is thermally fused to Flange #1. Hook #2 is available from Aplix SA France under the tradename SoftGrip® Gen2. The hook-flange laminate is made as disclosed in U.S. Pat. No. 10,618,204 and comprised of polypropylene, and have approximately 1,800 hooks per square inch.

HOOK #3: Hook #3 includes a 107 gsm hook (additionally a 25 gsm adhesive coating on the backing) that is available from 3M USA under the tradename KN2570. The hooks are described by 3M as micro-replicated oval-capped mushroom hooks, comprised of polypropylene, and have approximately 2,300 hooks per square inch.

### Landing Zone Materials:

LANDING ZONE #1: Landing Zone #1 includes a 35 gsm nonwoven that is available from PFN Znojmo, Czech Republic under tradename PFN: 32 35 00 00 05 00-70/30. The nonwoven is made of polypropylene and has SSS structure, i.e., has three layers spunbond, spunbond, spunbond, respectively. The nonwoven is thermally bonded and has “wave” shape bond pattern that is oriented transverse to the machine direction of the nonwoven.

LANDING ZONE #2: Landing Zone #2 includes a 25 gsm nonwoven that is available from PFN Znojmo, Czech Republic under tradename PFN: 32 25 00 00 05 00-70/30. The nonwoven is made of polypropylene and has SSS structure, i.e., has three layers spunbond, spunbond, spunbond, respectively. The nonwoven is thermally bonded and has “wave” shape bond pattern that is oriented transverse to the machine direction of the nonwoven.

LANDING ZONE #3: Landing Zone #3 includes a 20 gsm nonwoven that is available from PFN Znojmo, Czech Republic under tradename PFN: 32 20 00 00 05 00-70/30. The nonwoven is made of polypropylene and has SSS structure, i.e. has three layers spunbond, spunbond, spunbond, respectively. The nonwoven is thermally bonded and has “wave” shape bond pattern that is oriented transverse to the machine direction of the nonwoven.

LANDING ZONE #4: Landing Zone #4 includes a 15 gsm nonwoven that is available from PFN Znojmo, Czech Republic under tradename PFN: 32 15 00 00 05 00-70/30. The nonwoven is made of polypropylene and has SSS structure, i.e., has three layers spunbond, spunbond, spunbond, respectively. The nonwoven is thermally bonded and has “wave” shape bond pattern that is oriented transverse to the machine direction of the nonwoven.

LANDING ZONE #5: Landing Zone #5 includes a 15 gsm nonwoven that is available from Fibertex under the tradename A10150GT. The nonwoven is made of polypropylene and has SSS structure, i.e., has three layers spunbond, spunbond, spunbond, respectively and includes additives to improve softness. The nonwoven is thermally bonded and has an oval shape bond pattern.

Examples 1-6 are examples in accordance with the present disclosure and comprise the fastener component and landing zone materials in Table 1. Comparative Example A is a commercially available HUGGIES Pull-Ups® (Lot #PA102410F16829615). Comparative Examples B-F are comparative examples and comprise the fastener components and landing zone materials in Table 1.

Examples 1-6 and Comparative Examples A-F were evaluated for Fastener Component Caliper and Total Fastener Caliper according to the Caliper Test Method as described herein, Average Peak Stress according to the Fastener Ultra Sensitive 3 Point Bending Method as described herein, and Peak Peel Force according to the Fastener Shear Test and Peel Strength Method as described herein. The results of these tests are shown in Table 1.

TABLE 1

| | | | Fastener | Total | Average | Peak |
|---|---|---|---|---|---|---|
| | | | Component | Fastener | Peak | Peel |
| | Fastener | | Caliper | Caliper | Stress | Strength |
| | Component | Landing Zone | mm | mm | N/cm | N/inch |
| Example 1 | Hook #1 | Landing Zone | 0.358 | 0.619 | 0.45 | 3.2 |
| | | #1 | | | | |
| Example 2 | Hook #1 | Landing Zone | 0.358 | 0.619 | 0.45 | 2.8 |
| | | #2 | | | | |
| Example 3 | Hook #1 | Landing Zone | 0.358 | 0.619 | 0.45 | 2.2 |
| | | #3 | | | | |
| Example 4 | Hook #1 | Landing Zone | 0.358 | 0.619 | 0.45 | 2.2 |
| | | #4 | | | | |
| Example 5 | Hook #1 | Landing Zone | 0.358 | 0.619 | 0.45 | 2.4 |
| | | #5 | | | | |
| Example 6 | Hook #2 | Landing Zone | 0.245 | 0.516 | 0.34 | 2.0 |
| | | #2 | | | | |
| Comparative | Huggies | Huggies Pull- | 0.289 | 1.379 | 1.32 | 1.7 |
| Example A | Pull-Ups ® | Ups ® | | | | |
| Comparative | Hook #3 | Landing Zone | 0.356 | 0.642 | 0.81 | 1.5 |
| Example B | | #1 | | | | |
| Comparative | Hook #3 | Landing Zone | 0.356 | 0.642 | 0.81 | 1.4 |
| Example C | | #2 | | | | |
| Comparative | Hook #3 | Landing Zone | 0.356 | 0.642 | 0.81 | 1.4 |
| Example D | | #3 | | | | |
| Comparative | Hook #3 | Landing Zone | 0.356 | 0.642 | 0.81 | 1.3 |
| Example E | | #4 | | | | |
| Comparative | Hook #3 | Landing Zone | 0.356 | 0.642 | 0.81 | 0.8 |
| Example F | | #5 | | | | |

It was surprisingly found that Hook #1 and Hook #2 of Examples 1-6 are thin and flexible, as demonstrated by a Total Fastener Caliper of 0.619 mm and 0.516 mm, respectively, and an Average Peak Stress of 0.45 N/cm and 0.34 N/cm, respectively, yet still exhibited a Peak Peel Strength of 2.0 N/inch or more with various landing zone materials. The fastener component of Comparative Example A is thicker and less flexible as compared to Hook #1 and Hook #2, as demonstrated by a Fastener Caliper of 1.38 mm and an Average Peak Stress of 1.32 N/cm, and exhibited a lower Peak Peel Strength of only 1.7 N/inch. Hook #3 of Comparative Examples B-F is thin and flexible, as demonstrated by a Fastener caliper of 0.642 mm and an Average Peak Stress of 0.81 N/cm, however, only exhibited a Peak Peel Strength of 0.8 to 1.5 N/inch. Without being limited by theory, it is believed that by having a Total Fastener Caliper of from about 0.3 mm to about 1.2 mm and/or an Average Peak Stress of from about 0.1 to 1.0 N/cm, and a Peak Peel Strength of about 2.0 N/inch or more, the fastener component can provide secure attachment with the landing zone while still delivering an underwear-like look and feel (i.e., soft and cloth-like).

### Average Decitex (Average-Dtex)

The Average Decitex Method is used to calculate the Average-Dtex on a length-weighted basis for elastic fibers present in an entire article, or in a specimen of interest extracted from an article. The decitex value is the mass in grams of a fiber present in 10,000 meters of that material in the relaxed state. The decitex value of elastic fibers or elastic laminates containing elastic fibers is often reported by manufacturers as part of a specification for an elastic fiber or an elastic laminate including elastic fibers. The Average-Dtex is to be calculated from these specifications if available. Alternatively, if these specified values are not known, the decitex value of an individual elastic fiber is measured by determining the cross-sectional area of a fiber in a relaxed state via a suitable microscopy technique such as scanning electron microscopy (SEM), determining the composition of the fiber via Fourier Transform Infrared (FT-IR) spectroscopy, and then using a literature value for density of the composition to calculate the mass in grams of the fiber present in 10,000 meters of the fiber. The manufacturer-provided or experimentally measured decitex values for the individual elastic fibers removed from an entire article, or specimen extracted from an article, are used in the expression below in which the length-weighted average of decitex value among elastic fibers present is determined.

The lengths of elastic fibers present in an article or specimen extracted from an article is calculated from overall dimensions of and the elastic fiber pre-strain ratio associated with components of the article with these or the specimen, respectively, if known. Alternatively, dimensions and/or elastic fiber pre-strain ratios are not known, an absorbent article or specimen extracted from an absorbent article is disassembled and all elastic fibers are removed. This disassembly can be done, for example, with gentle heating to soften adhesives, with a cryogenic spray (e.g., Quick-Freeze, Miller-Stephenson Company, Danbury, CT), or with an appropriate solvent that will remove adhesive but not swell, alter, or destroy elastic fibers. The length of each elastic fiber in its relaxed state is measured and recorded in millimeters (mm) to the nearest mm.

### Calculation of Average-Dtex

For each of the individual elastic fibers fᵢ of relaxed length Lᵢ and fiber decitex value dᵢ (obtained either from the manufacturer's specifications or measured experimentally) present in an absorbent article, or specimen extracted from an absorbent article, the Average-Dtex for that absorbent article or specimen extracted from an absorbent article is defined as:

$$Average - Dtex = \frac{{\sum }_{i = 1}^{n} ( {L}_{i} \times {d}_{i} )}{{\sum }_{i = 1}^{n} {L}_{i}}$$

where n is the total number of elastic fibers present in an absorbent article or specimen extracted from an absorbent article. The Average-Dtex is reported to the nearest integer value of decitex (grams per 10 000 m).

If the decitex value of any individual fiber is not known from specifications, it is experimentally determined as described below, and the resulting fiber decitex value(s) are used in the above equation to determine Average-Dtex.

### Experimental Determination of Decitex Value for a Fiber

For each of the elastic fibers removed from an absorbent article or specimen extracted from an absorbent article according to the procedure described above, the length of each elastic fiber Lₖ in its relaxed state is measured and recorded in millimeters (mm) to the nearest mm. Each elastic fiber is analyzed via FT-IR spectroscopy to determine its composition, and its density ρₖ is determined from available literature values. Finally, each fiber is analyzed via SEM. The fiber is cut in three approximately equal locations perpendicularly along its length with a sharp blade to create a clean cross-section for SEM analysis. Three fiber segments with these cross sections exposed are mounted on an SEM sample holder in a relaxed state, sputter coated with gold, introduced into an SEM for analysis, and imaged at a resolution sufficient to clearly elucidate fiber cross sections. Fiber cross sections are oriented as perpendicular as possible to the detector to minimize any oblique distortion in the measured cross sections. Fiber cross sections may vary in shape, and some fibers may consist of a plurality of individual filaments. Regardless, the area of each of the three fiber cross sections is determined (for example, using diameters for round fibers, major and minor axes for elliptical fibers, and image analysis for more complicated shapes), and the average of the three areas aₖ for the elastic fiber, in units of micrometers squared (μm²), is recorded to the nearest 0.1 μm². The decitex dₖ of the kth elastic fiber measured is calculated by:

$${d}_{k} = 10000 m \times {a}_{k} \times {ρ}_{k} \times {10}^{- 6}$$

where dₖ is in units of grams (per calculated 10,000 meter length), aₖ is in units of μm², and ρₖ is in units of grams per cubic centimeter (g/cm³). For any elastic fiber analyzed, the experimentally determined Lₖ and dₖ values are subsequently used in the expression above for Average-Dtex.

### Average-Strand-Spacing

Using a ruler calibrated against a certified NIST ruler and accurate to 0.5 mm, measure the distance between the two distal strands within a section to the nearest 0.5 mm, and then divide by the number of strands in that section—1
Average-Strand-Spacing=d/(n−1) where n>1
report to the nearest 0.1 mm.

### Average-Pre-Strain

The Average-Pre-Strain of a specimen are measured on a constant rate of extension tensile tester (a suitable instrument is the MTS Insight using Testworks 4.0 Software, as available from MTS Systems Corp., Eden Prairie, MN) using a load cell for which the forces measured are within 1% to 90% of the limit of the cell. Articles are conditioned at 23° C.±2° C. and 50%±2% relative humidity for 2 hours prior to analysis and then tested under the same environmental conditions.

Program the tensile tester to perform an elongation to break after an initial gage length adjustment. First raise the cross head at 10 mm/min up to a force of 0.05 N. Set the current gage to the adjusted gage length. Raise the crosshead at a rate of 100 mm/min until the specimen breaks (force drops 20% after maximum peak force). Return the cross head to its original position. Force and extension data is acquired at a rate of 100 Hz throughout the experiment.

Set the nominal gage length to 40 mm using a calibrated caliper block and zero the crosshead. Insert the specimen into the upper grip such that the middle of the test strip is positioned 20 mm below the grip. The specimen may be folded perpendicular to the pull axis, and placed in the grip to achieve this position. After the grip is closed the excess material can be trimmed. Insert the specimen into the lower grips and close. Once again, the strip can be folded, and then trimmed after the grip is closed. Zero the load cell. The specimen should have a minimal slack but less than 0.05 N of force on the load cell. Start the test program.

From the data construct a Force (N) verses Extension (mm). The Average-Pre-Strain is calculated from the bend in the curve corresponding to the extension at which the nonwovens in the elastic are engaged. Plot two lines, corresponding to the region of the curve before the bend (primarily the elastics), and the region after the bend (primarily the nonwovens). Read the extension at which these two lines intersect, and calculate the % Pre-Strain from the extension and the corrected gage length. Record as % Pre-strain 0.1%. Calculate the arithmetic mean of three replicate samples for each elastomeric laminate and Average-Pre-Strain to the nearest 0.1%.

### Ultra Sensitive 3 Point Bending Method

The Ultra Sensitive 3 Point Bending Method is used to measure peak bending force of a male fastener component of a sample refastenable pant-style diaper. Specimens are measured using constant-rate-of-extension tensile tester outfitted with specific 3 point bend tooling, described further below, that allows unusually high signal-to-noise when measuring low bending forces. Testing is performed at 23±3° C. and 50±2% relative humidity, and sample articles to be tested are conditioned for at least two hours prior to testing in these same environmental conditions.

### Sample Preparation

Six specimens of the male fastener components are excised from three like sample articles. On each of the three like sample articles, one specimen is excised from the male fastener component on the wearer's left side and one specimen from the male fastener component on the wearer's right side. Each specimen of male fastener component is taken at the approximate longitudinal center of the male fastener component. Each specimen is cut so that its lateral edges are coincident with the lateral edges of the male fastener component and such that it is 20 mm in longitudinal extent (again, taken from the approximate longitudinal center of the male fastener component). If the male fastener components are constructed with multi-stripe features, the test specimens are cut at the outer-most lateral edges of the multi-stripe features. The lateral width of each specimen is measured to the nearest 0.1 mm and recorded.

### Apparatus

A universal constant rate of extension tensile tester (such as MTS Alliance, MTS Systems Corp., Eden Prairie, MN, or equivalent) equipped with a load cell (e.g. 5N limit) appropriate for the forces being measured is used for this method. The tensile tester configured with ultra sensitive 3 point bend tooling designed to maximize the force signal to noise ratio when testing materials with very low bending forces. The force signal is maximized by using a high sensitivity load cell (e.g. 5 N), using a small span (load is proportional to the span cubed), and using a wide specimen width (total measured load is directly proportional to width). The fixture is designed such that the bending measurement is performed in tension, allowing the fixture mass to be kept to a minimum. Noise in the force signal is minimized by holding the load cell stationary to reduce mechanical vibration and inertial effect and by making the mass of the fixture attached to the load cell as low as possible.

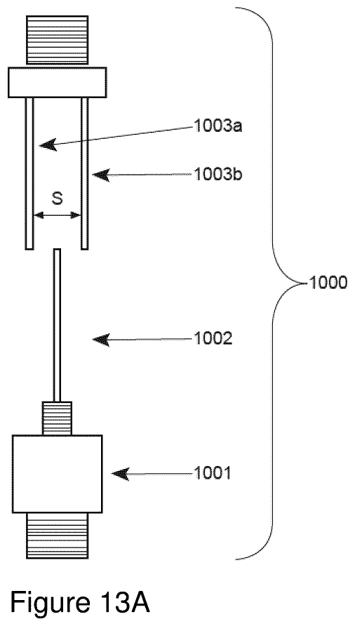

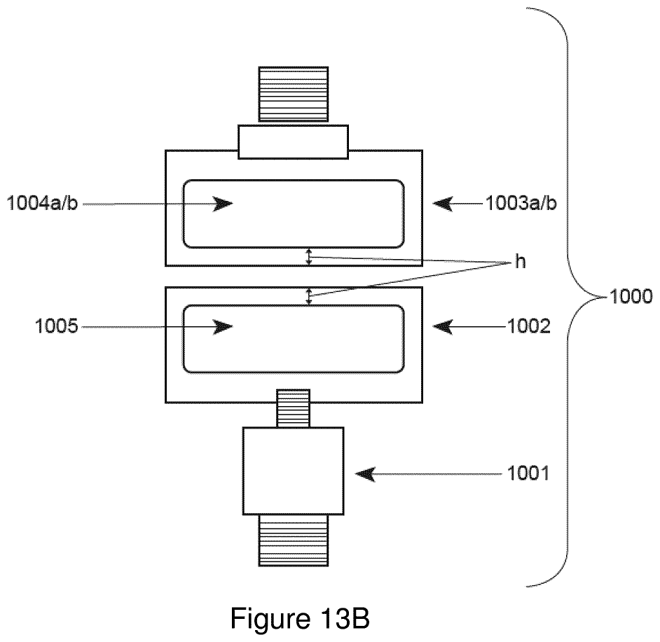

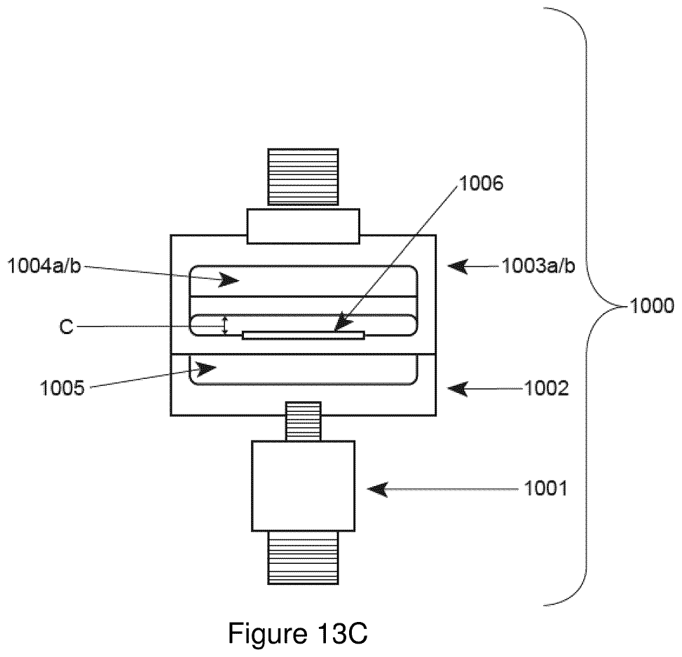

Referring to FIGS. 13A-13C, the load cell 1001 is mounted on the stationary crosshead of the universal test frame. The fixture 1000 consists of three thin blades constructed of aluminum. Each blade has a thickness of 1.0 mm, rounded edges and a length that can accommodate a bending width of up to 100 mm. Each of the blades has a cavity 1004a and 1004b (outside blades) and 1005 (central blade) cut out to create a height, h, of 5 mm of blade material along their horizontal edges. The two outside blades 1003a and 1003b are mounted horizontally to the moveable crosshead of the universal test frame, aligned parallel to each other, with their horizontal edges vertically aligned. The span, s, between the two outside blades 1003a and 1003b is 5 mm±0.1 mm (inside edge to inside edge). The central blade 1002 is mounted to the load cell on the stationary crosshead of the universal test frame. When in place, the central blade 1002 is parallel to the two outside blades 1003a and 1003b and centered at the midpoint between the outside blades 1003a and 1003b. The blade fixtures include integral adapters appropriate to fit the respective positions on the universal test frame and lock into position such that the horizontal edges of the blades are orthogonal to the motion of the crossbeam of the universal test frame.

### Measurement Procedure

Prior to loading a specimen 1006, the outside blades 1003a and 1003b are moved towards and then past central blade 1002 until there is approximately a 3 mm clearance, c, between the inner horizontal edges of cavities 1004a and 1004b in the outside blades 1003a and 1003b and the inner horizontal edge of cavity 1005 in the central blade 1002 (see FIG. 13C). A specimen 1006 is placed within clearance c such that it spans the inner horizontal edges of cavities 1004a and 1004b in the outside blades 1003a and 1003b, oriented such that the longitudinally running lateral edges of the specimen are perpendicular to the horizontal edges of the blades and the fastening surface of the specimen is facing up. The specimen 1006 is centered between the outside blades 1003a and 1003b. The outside blades 1003a and 1003b are moved in a direction opposite of the stationary crosshead until the inner horizontal edge of cavity 1005 in the central blade 1002 touches the top surface of the specimen 1006.

The tensile tester is configured such that the moveable crosshead moves away from the stationary crosshead at a rate of 1.0 mm/s. Crosshead movement begins with the specimen 1006 lying flat and undeflected on the outer blades 1003a and 1003b as described above, continues with the inner horizontal edge of cavity 1005 in the central blade 1002 coming into contact with the top surface of the specimen 1006, and further continues for an additional 4 mm of crosshead movement. The crosshead stops at 4 mm and then immediately returns to zero at a speed of 1.0 mm/s. Force (N) and displacement (mm) are collected at 50 Hz throughout. This specimen placement and measurement procedure is performed on all six specimens.

### Analysis and Reporting

From the bending data for each of the six specimens, the peak force is determined and recorded for each specimen to the nearest 0.001 N. For each specimen, the peak stress for that specimen is calculated by dividing peak force by the specimen width (measured and recorded earlier) and is recorded to the nearest 0.01 N/cm. The arithmetic mean of the peak stress for each of the six specimens is calculated and reported as the Average Peak Stress, in N/cm, to the nearest 0.01 N/cm.

### Caliper Test Method

The Caliper Test Method is used to measure the caliper of generally flat materials such as waist region substrate laminates including fastener components and waist region substrate proximal to fastener components. In this method, specimens of material of interest are measured horizontally and on a hard, flat surface using a digital caliper gauge fitted with a circular foot. Testing is performed at 23±3° C. and 50±2% relative humidity, and sample articles to be tested are conditioned for at least two hours prior to testing in these same environmental conditions.

### Sample Preparation

Three like refastenable diapers are separated at their fastener components so as to separate the first and second waist regions. On each diaper, fastener components to be measured and appearing on either the first or second waist regions are identified. On either the first or second waist region (depending on which contains the fastener components of interest to be measured), two fastener components will generally be present (one each toward either lateral edge of the waist region). Two rectangles of waist region are excised, each of which contains one fastener component. The rectangle has the same longitudinal extent as the fastener component, is bounded laterally outboard by the outboard lateral edge of the fastener component, and is bounded laterally inboard by a line 15 mm inboard of the inboard lateral edge of the fastener component. Among the three diapers, six specimens are prepared in this way.

### Apparatus and Procedure

Suitable apparatus enables caliper to be measured between an upper metal circular flat foot 10.0 mm in diameter and hard lower surface such as a granite base. The upper circular flat foot imparts 7.2 kPa of downward pressure during caliper measurement and is attached to a linear caliper gauge with at least 0.001 mm accuracy.

For each of the six specimens, a specimen is first oriented such that the center point (both longitudinal and lateral) of the fastener component is centered with the circular measuring foot, and the foot is then gently lowered (imparting no discernible stress beyond the static pressure of the foot) onto the fastener component. After a dwell time of 10 s, a caliper reading is recorded for that specimen as the fastener component laminate caliper to the nearest 0.001 mm. The foot is then lifted, and the specimen is shifted laterally such that the circular foot is centered on the 15 mm strip of proximal waist region substrate excised along with the fastener component. The foot is then gently lowered (imparting no discernible stress beyond the static pressure of the foot) onto the proximal waist region substrate. After a dwell time of 10 s, a caliper reading is recorded for that specimen as the proximal waist region laminate caliper to the nearest 0.001 mm. The difference between the fastener component laminate caliper and the proximal waist region laminate caliper is then calculated and recorded to the nearest 0.001 mm as the fastener component caliper. This procedure and calculation are repeated for each of the six specimens.

### Reporting

The arithmetic mean of the six individual specimen fastener component calipers is calculated and reported 0.001 mm as the Fastener Component Caliper for the fastener component of interest. The arithmetic mean of the six individual specimen fastener component laminate calipers is calculated and reported 0.001 mm as the Total Fastener Caliper for the fastener component of interest.

### Fastener Shear Test and Peel Strength

The Fastener Shear and Peel Strength Test Method is used to measure first shear properties and then, conditionally, the 180-degree peel strength of fastener components of a refastenable pant-style diaper of interest. The Fastener Shear portion of the method is a pass/fail assessment in which a specimen of affixed fastener components are subjected to a 500-gf shear force. If all specimens pass this assessment, they are then subjected to a 180-degree peel method on using a constant-rate-of-extension tensile tester. Testing is performed at 23±3° C. and 50±2% relative humidity, and sample articles to be tested are conditioned for at least two hours prior to testing in these same environmental conditions. All references to lateral and longitudinal in this method correspond to the appearance of element in a finished diaper or, after excision, the direction that would apply were the excised portion still in place on the finished diaper.

### Sample Preparation

Three like articles of a refastenable pant-style diaper of interest are used. Six specimen pairs are taken from these three articles, one pair corresponding to the fastening side features on each of the wearer's right or left side of each of the three articles. (A fastening side feature is the unitary feature formed by a fastener component on the first waist region and a fastener component on the second waist region in a finished article.) That is, any fastening side feature at a wearer's right or left side comprises two fastener components, one from the first waist region and one from the second waist region, in contact. A specimen pair consists of two portions, one taken from each of the two fastener components as described below in a manner that preserves their original overlap and registration in the fastening side feature.

A specimen pair is created by first identifying a fastening side feature and marking both longitudinally running lateral edges of the fastening side feature as well as the longitudinal extrema of the side feature. Next, the first and second waist region joined at the fastening side feature are separated. From each of the first and second waist regions that formed the fastening side feature, a laterally running strip that is 1.0 inch in longitudinal extent is cut. The strip is centered longitudinally with regard to the overall fastening side feature longitudinal extent and extends from the marked laterally inboard edge of the fastening side feature to the proximal longitudinally running lateral edge of the waist region. These two strips (one each from the first and second waist regions) form a specimen pair.

The two strips of the specimen pair are then mounted on bilayer of masking tape (3M 201+masking tape, 3M, Maplewood, Minnesota, USA, or equivalent) of the same width as the longitudinal length (1.0 inch) of the strips. For each strip of the specimen pair, a piece of masking tape 100 mm in length is cut. To the adhesive surface of each strip is affixed one strip of the specimen pair. The strip is placed in so as to be centered along the 100-mm dimension of the tape and such that the side of the strip that took part in the refastenable attachment is facing away from the adhesive. The strip is pressed firmly onto the tape to adhere it. Following this, for each strip of the specimen pair, the open adhesive of the tape is closed with a downward facing piece of tape wherever necessary. That is, on either side of where the strip of the specimen pair is adhered to the 100-mm long piece of tape, a piece of tape is cut and affixed such that adhesive sides face each other and such that all exposed adhesive is covered while no portion of the strip of the specimen pair is obscured. Henceforth in this method, “laterally inboard edge of strip” means the extremum of the overall tape and strip assembly nearest to the laterally inboard edge of the affixed strip, and “laterally outboard edge of strip” means the extremum of the overall tape and strip assembly nearest to the laterally outboard edge of the affixed strip.

### Shear Test Procedure

The shear test is carried out on all like specimen pairs, prepared as described above. The specimen pairs in the shear test are refastened with specific objective of being oriented in the same fashion as they were in the pre-fastened diaper. The laterally inboard edge of the strip excised from the second waist region portion is placed in a fixed clamp. The laterally inboard edge of the strip excised from the first waist region portion is affixed (for example, using a clamp) to a 500-g mass. The two strips are then superposed in the same orientation in which they were in the pre-fastened diaper. The 500-g mass is temporarily supported (for example, by hand), and the unmasked portions of the strips containing the fastener components are brought together and pressed firmly between thumb and forefinger for 3 seconds, with care to distribute the finger-pinch across the width of the fastener.

The 500-g mass is then immediately but gently lowered so as not to exert any discernible additional force beyond the 500 gf static force, and the fastened strips are subjected to this force for 5 seconds. If the strips separate during 5 seconds, the specimen pair is recorded as having failed. If the strips do not separate during 5 seconds, the specimen pair is recorded as having passed.

This same procedure is repeated for all six specimen pairs. If any of the six specimen pairs fail, the test is over, and no peel measurement is performed and peel force is deemed to be zero Newtons (0 N). If all six specimen pairs pass, all six are then peeled according to the procedure below.

### Peel Test Procedure

If all six specimen pairs successfully pass the shear test procedure above, they are then immediately subjected to a 180-degree peel using a constant-rate-of-extension tensile tester. The objective is to measure the force associated with peeling apart the fastener components. For each specimen pair, to be measured, the tensile tester is equipped with clamps at least 1.0 inch in width, and the initial gauge length is set to 50 mm. Into the lower clamp is placed the laterally inboard-most edge of the strip from the second waist region—the same that was placed in the fixed clamp in the shear test procedure. Into the upper clamp is placed the laterally outboard-most edge of the strip from the first waist region. The specimen pair is positioned with enough tension such that the force reading at the start of the test is 0.2±0.1 N. The clamps are then separated at 305 mm/min for an incremental displacement of at least 40 mm or until the fastener components are completely separated. Force versus displacement data are recorded at 50 Hz throughout the tensile pull. The procedure is repeated for each of the six specimen pairs.

For each of the six specimen pairs, the peak force of the peel is determined and recorded to the nearest 0.1 N. For each of the six specimen pairs, the determined peak force is divided by the longitudinal extent of that specimen pair to establish a corresponding peak strength in N/inch. The arithmetic mean of the peak strength among the six specimen pairs is calculated and reported in N/inch to the nearest 0.1 N/inch as the Peak Peel Strength of the fastener components of the refastenable diaper of interest.

### Whole Article Force Method

The Whole Article Force Test Method is used to measure the Full Stretch Circumference and the Fit Circumference Force of a refastenable pant-style diaper with pre-fastened fastening side features. Specimens of a diaper of interested are mounted on a set of fixture bars on a tensile tester that enable the waist circumference of the diaper to be stretched as if worn, and Full Stretch Circumference and Fit Circumference Force are determined from force versus displacement data. Testing is performed at 23±3° C. and 50±2% relative humidity, and sample articles to be tested are conditioned for at least two hours prior to testing in these same environmental conditions.

### Apparatus

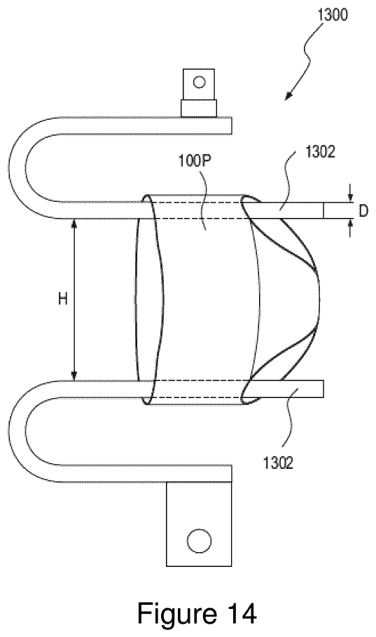

A constant rate of extension tensile tester is equipped with fixture bars 1302 as shown in FIG. 14. Each fixture bar has a round cross section 10.0 mm in diameter and is fashioned from a material that is rigid under the typical for circumferential waist extension of a diaper. The surface of each fixture bar is sufficiently smooth so as not to restrict sliding of the diaper over the surface during stretching. The horizontal bar sections (perpendicular to the axis of crosshead separation of the tensile tester) are sufficient in length to accommodate the entire pre-fastened fastening side features of the diaper and remain parallel and in the same vertical plane throughout the test procedure. At any crosshead position and the gauge circumference is defined by

$$Gauge circumference = 2 H + 2 D + π D$$

where H and D are the separation between horizontal bars and the bar diameter, respectively, as shown in FIG. 14.

### Procedure

The specimen diaper is placed onto the upper horizontal bar such that the bar passes through the waist opening and one leg opening of the diaper specimen and such that the pre-fastened refastenable side feature remains fully closed. The crosshead is raised until the diaper specimen hangs above the lower bar but does not touch the lower bar. The load cell is then tared, and the crosshead is lowered to enable the easy insertion of the lower bar through the waist opening and other leg opening without stretching the diaper specimen. The diaper specimen is adjusted at this point such that the longitudinal centerline of both the front and rear of the diaper specimen is located halfway between the upper and lower bars. The longitudinal center of the side portions in contact with the bars are situated on the axis between the upper and lower crosshead attachment points and load cell.

The crosshead is then raised slowly while the diaper specimen is stabilized by hand (as necessary to maintain the required position) until the force is 0.075±0.025 N. The crosshead then is moved upward (separating the bars) at 254.0 mm/min until a final load of 19.61 N is reached, at which point the crosshead immediately is returned to its initial position at 254.0 mm/min. Data are acquired throughout at 50 Hz. The gauge circumference at maximum load (19.61 N) is determined and recorded to the nearest 0.1 mm as the full stretch circumference for that diaper specimen. The load corresponding to 75% of the full stretch circumference on the latter, returning half of the tensile tester movement is determined and recorded as the fit circumference force to the nearest 0.01 N for that diaper specimen.

Five like diaper specimens of a diaper of interest are analyzed as specified. The arithmetic mean of the full stretch circumferences of among the five diaper specimens is calculated and reported to the nearest 0.1 mm as the Full Stretch Circumference of the diaper. The arithmetic mean of the fit circumference force of among the five diaper specimens is calculated and reported to the nearest 0.01 N as the Fit Circumference Force of the diaper. If the fastener components of the diaper specimen remain attached to the opposing waist region, the specimen is reported as “passing” the Whole Article Force Test.

### Combinations

Paragraph A. An absorbent article comprising:
   a chassis comprising a topsheet, a backsheet, and an absorbent core positioned between the topsheet and the backsheet, the chassis further comprising a first chassis end region, a second chassis end region longitudinally separated from the first chassis end region by a crotch region, a first chassis side region, and a laterally opposing second chassis side region;
   a first waist region joined to the first chassis end region, wherein the first waist region comprises a first end region and a second end region laterally separated from the first end region by a central region, the first waist region further comprising a first surface and a second surface opposite the first surface;
   a second waist region joined to the second chassis end region, wherein the second waist region comprises a first end region and a laterally opposed second end region;
   wherein the first end region of the first waist region comprises a first fastener component disposed on the first surface, wherein the first fastener component is refastenably connected with the first end region of the second waist region;
   wherein the second end region of the first waist region comprises a second fastener component disposed on the first surface, wherein the second fastener component is refastenably connected with the second end region of the second waist region;
   wherein the first and second fastener components each comprise a plurality of hooks;
   wherein the first waist region comprises a Total Fastener Caliper measured from the second surface of the first waist region to a distal end of the plurality of hooks of from 0.3 mm to 1.2 mm;
   wherein the fastener components exhibit a Peak Peel Strength of 2.0 N/inch or more.
Paragraph B. The absorbent article of Paragraph B, wherein the first waist region has a basis weight of from 25 gsm to 90 gsm.
Paragraph C. The absorbent article of Paragraphs A or B, wherein a first portion of the first waist region extends laterally outward from the first chassis side region forming a first side panel, wherein the first fastener component is disposed on a first end region of the first side panel.
Paragraph D. The absorbent article of Paragraph C, wherein a second portion of the first waist region extends laterally outward from the second chassis side region forming a second side panel, wherein the second fastener component is disposed on a first end region of the second side panel.
Paragraph E. The absorbent article of Paragraph A, wherein the first waist region extends laterally across the entire chassis, wherein the first chassis end region is connected with the central region of the first waist region.
Paragraph F. The absorbent article of Paragraph E, wherein a portion of the first waist region comprises a first elastic belt, the first elastic belt comprising a first end region and a laterally opposed second end region.
Paragraph G. The absorbent article of Paragraph F, wherein the second waist region comprises a second elastic belt.
Paragraph H. The absorbent article of Paragraph F, wherein the first waist region comprises a first flange, wherein the first flange comprises a first lateral end region and a second lateral end region, wherein the first lateral end region of the first flange is joined to the first end region of the first elastic belt, and wherein the first fastener component is positioned on the second lateral end region of the first flange.
Paragraph I. The absorbent article of Paragraph H, wherein the first waist region comprises a second flange, wherein the second flange comprises a first lateral end region and a second lateral end region, wherein the first lateral end region of the second flange is joined to the second end region of the first elastic belt, and wherein the second fastener component is positioned on the second lateral end region of the second flange.
Paragraph J. The absorbent article of any of Paragraphs A to I, wherein the first and second fastener components each comprise a plurality of hooks extending from a base.
Paragraph K. The absorbent article of Paragraph A, wherein the first and second fastener components each comprise a plurality of hooks integrally formed from a material of the first waist region.
Paragraph L. The absorbent article of Paragraph F or G, wherein the first and second elastic belt each comprise a first substrate, a second substrate, and an elastic material positioned between and connected with the first substrate and the second substrate.
Paragraph M. The absorbent article of Paragraph L, wherein the second substrate comprises a nonwoven having a basis weight of from 15 to 35 gsm.
Paragraph N. The absorbent article of Paragraph I, wherein the first and second flanges each comprises a first end edge and a longitudinally opposed second end edge, wherein the first and second fastener components each comprise a first end edge and a longitudinally opposed second end edge, wherein the first end edge of the fastener components is positioned longitudinally inboard of the first end edge of the flanges by a distance of from 2 mm to 8 mm.
Paragraph O. The absorbent article of any of Paragraphs A to N, wherein the fastener component comprises a plurality of hooks having a hook density of from about 1300 to about 2300 hooks per square inch.
Paragraph P. The absorbent article of any of Paragraphs A to O, wherein the first end region and the second end region of the first waist region exhibit an Average Peak Stress of 0.10 to 1.0 N/cm measured at a position within the fastener component.
Paragraph Q. The absorbent article of any of Paragraphs A to P, wherein the first and second fastener components comprise a laminate comprising layers, wherein the layers are bonded together by extrusion bonding, adhesive, mechanical bonding, thermal fusion, and/or ultrasonic welding.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent article comprising:
a chassis comprising a topsheet, a backsheet, and an absorbent core positioned between the topsheet and the backsheet, the chassis further comprising a first chassis end region, a second chassis end region longitudinally separated from the first chassis end region by a crotch region, a first chassis side region, and a laterally opposing second chassis side region;
a first waist region joined to the first chassis end region, wherein the first waist region comprises a first end region and a second end region laterally separated from the first end region by a central region, the first waist region further comprising a first surface and a second surface opposite the first surface;
a second waist region joined to the second chassis end region, wherein the second waist region comprises a first end region and a laterally opposed second end region;
wherein the first end region of the first waist region comprises a first fastener component disposed on the first surface, wherein the first fastener component is refastenably connected with the first end region of the second waist region;
wherein the second end region of the first waist region comprises a second fastener component disposed on the first surface, wherein the second fastener component is refastenably connected with the second end region of the second waist region;
wherein the first and second fastener components each comprise a plurality of hooks;
wherein the first waist region comprises a total fastener caliper measured from the second surface of the first waist region to a distal end of the plurality of hooks of from about 0.3 mm to about 1.2 mm;
wherein the fastener components exhibit a Peak Peel Force of about 2.0 N/inch or more.

2. The absorbent article of claim 1, wherein the first waist region has a basis weight of from about 25 gsm to about 90 gsm.

3. The absorbent article of claim 1, wherein a first portion of the first waist region extends laterally outward from the first chassis side region forming a first side panel, wherein the first fastener component is disposed on a first end region of the first side panel.

4. The absorbent article of claim 3, wherein a second portion of the first waist region extends laterally outward from the second chassis side region forming a second side panel, wherein the second fastener component is disposed on a first end region of the second side panel.

5. The absorbent article of claim 1, wherein the first waist region extends laterally across the entire chassis, wherein the first chassis end region is connected with the central region of the first waist region.

6. The absorbent article of claim 5, wherein a portion of the first waist region comprises a first elastic belt, the first elastic belt comprising a first end region and a laterally opposed second end region.

7. The absorbent article of claim 6, wherein the second waist region comprises a second elastic belt.

8. The absorbent article of claim 6, wherein the first waist region comprises a first flange, wherein the first flange comprises a first lateral end region and a second lateral end region, wherein the first lateral end region of the first flange is joined to the first end region of the first elastic belt, and wherein the first fastener component is positioned on the second lateral end region of the first flange.

9. The absorbent article of claim 8, wherein the first waist region comprises a second flange, wherein the second flange comprises a first lateral end region and a second lateral end region, wherein the first lateral end region of the second flange is joined to the second end region of the first elastic belt, and wherein the second fastener component is positioned on the second lateral end region of the second flange.

10. The absorbent article of claim 1, wherein the first and second fastener components each comprise a plurality of hooks extending from a base.

11. The absorbent article of claim 1, wherein the first and second fastening components each comprise a plurality of hooks integrally formed from a material of the first waist region.

12. The absorbent article of claim 7, wherein the first and second elastic belt comprise a first substrate, a second substrate, and an elastic material positioned between and connected with the first substrate and the second substrate.

13. The absorbent article of claim 12, wherein the second substrate comprises a nonwoven having a basis weight of from about 15 to about 35 gsm.

14. The absorbent article of claim 9, wherein the first and second flanges each comprises a first end edge and a longitudinally opposed second end edge, wherein the first and second fastener components each comprise a first end edge and a longitudinally opposed second end edge, wherein the first end edge of the fastener components is positioned longitudinally inboard of the first end edge of the flanges by a distance of from about 2 mm to about 8 mm.

15. The absorbent article of claim 1, wherein the fastener component comprises a plurality of hooks having a hook density of from about 1300 to about 2300 hooks per square inch.

16. An absorbent article comprising:
a chassis comprising a topsheet, a backsheet, and an absorbent core positioned between the topsheet and the backsheet, the chassis further comprising a first chassis end region, a second chassis end region longitudinally separated from the first chassis end region by a crotch region, a first chassis side region, and a laterally opposing second chassis side region;
a first waist region joined to the first chassis end region, wherein the first waist region comprises a first end region and a second end region laterally separated from the first end region by a central region, the first waist region further comprising a first surface and a second surface opposite the first surface;
a second waist region joined to the second chassis end region, wherein the second waist region comprises a first end region and a laterally opposed second end region;
wherein the first end region of the first waist region comprises a first fastener component disposed on the first surface, wherein the first fastener component is refastenably connected with the first end region of the second waist region;
wherein the second end region of the first waist region comprises a second fastener component disposed on the first surface, wherein the second fastener component is refastenably connected with the second end region of the second waist region;
wherein the first and second fastener components each comprise a plurality of hooks;
wherein the fastener components exhibit a Peak Peel Force of 2.0 N/inch or more;
wherein the first end region and the second end region of the first waist region exhibit a Bending Stress of 0.10 to 1.0 N/cm measured at a position within the fastener component.

17. The absorbent article of claim 16, wherein the first waist region extends laterally across the entire chassis, wherein the first chassis end region is connected with the central region of the first waist region.

18. The absorbent article of claim 17, wherein a portion of the first waist region comprises a first elastic belt, the first elastic belt comprising a first end region and a laterally opposed second end region.

19. The absorbent article of claim 18, wherein the first waist region comprises a first flange, wherein the first flange comprises a first lateral end region and a second lateral end region, wherein the first lateral end region of the first flange is joined to the first end region of the first elastic belt, and wherein the first fastener component is positioned on the second lateral end region of the first flange.

20. The absorbent article of claim 19, wherein the first waist region comprises a second flange, wherein the second flange comprises a first lateral end region and a second lateral end region, wherein the first lateral end region of the second flange is joined to the second end region of the first belt, and wherein the second fastener component is positioned on the second lateral end region of the second flange.
